(19) United States
(12) Patent Application Publication
(10) Pub. No.: US 2024/0409642 A1
(43) Pub. Date: Dec. 12, 2024

(54) INHIBITORS FOR USE IN PREVENTION- AND/OR ALLEVIATION OF HEART FIBROSIS

(71) Applicants: Syddansk Universitet, Odense M (DK); Region Syddanmark, Vejle (DK)

(72) Inventors: Ditte Caroline ANDERSEN, Odense SV (DK); Charlotte Harken JENSEN, Odense SV (DK)

(21) Appl. No.: 18/570,263

(22) PCT Filed: Jun. 16, 2022
(86) PCT No.: PCT/EP2022/066489

§ 371 Date: Dec. 14, 2023

(30) Foreign Application Priority Data
Jun. 21, 2021 (EP) 21180586.6

(51) Int. Cl. C07K 16/28; A61K 31/713; A61K 39/00; C12N 15/113; G01N 33/68

(52) U.S. Cl. CPC C07K 16/2839; A61K 31/713; C12N 15/1138; G01N 33/6893; A61K 2039/505; G01N 2800/32; G01N 2800/52

(57) ABSTRACT

Inhibitors for use in preventing and/or alleviation of increased fibrotic scar tissue formation caused by a disease or a complication of the heart, such as a myocardial infarction or surgery. The inhibitors may inhibit activity of the Delta Like Non-Canonical Notch Ligand 1 (Dlk1) protein or the integrin beta-8 (Itgb8) protein.

### CROSS-REFERENCE TO RELATED APPLICATIONS

The present application claims priority to PCT application serial no. PCT/EP2022/066489 filed Jun. 16, 2022, which claims priority to European patent application serial no. 21180586.6, filed Jun. 21, 2021, each herein incorporated by reference in their entireties.

### SEQUENCE LISTING

The present application contains a Sequence Listing which is submitted electronically as an ASCII text file and is incorporated by reference in its entirety. Said ASCII text file was created on May 21, 2024, is named PMV112US_ASCIISequenceListing.txt and 7,615 bytes in size.

### TECHNICAL FIELD OF THE INVENTION

The present invention relates to prevention and alleviation of increased fibrotic scar tissue formation in the heart using protein inhibitors. In particular, the present invention relates to inhibitors of Delta Like Non-Canonical Notch Ligand 1 (Dlk1) protein or integrin beta-8 (Itgb8) protein.

### BACKGROUND OF THE INVENTION

Scar tissue formation is caused by fibroblasts that are activated to proliferate and secrete substantial amounts of extracellular matrix (ECM) including collagen deposition. Extensive scar tissue formation in the heart may lead to a reduced function of the heart's ability to circulate blood in the body. Formation of scar tissue may be caused or activated by different conditions of the heart, such as a heart attack, disease, surgery or cancer. A heart attack, i.e. a myocardial infarction (MI), results in extensive fibrotic scar formation that stiffens the heart muscle and reduces cardiac function eventually leading to heart failure, a major cause of death worldwide. Fate mapping studies underscore that MI-remodelling cardiac fibroblasts are derived from the peri-/epicardium, where epicardium derived cells (EPDCs) proliferate and then undergo epithelial to mesenchymal transition (EMT) differentiating into mainly cardiac fibroblasts and smooth muscle that migrate and colonize the myocardium or stay in close proximity to the epicardium. Signalling pathways involving Transforming growth factor β (TGFβ), Wnt/β-catenin, and platelet-derived growth factor (PDGF) have been shown to mediate EMT and migration of EPDCs during heart development and seems to be reactivated after MI. Moreover, the peri-/epicardium itself seems to work as a paracrine organ involved in cardiac fibrosis after MI. Proteins in the peri-/epicardium that facilitate EMT and fibrosis is of high interest as therapeutic targets for inhibition of cardiac fibrosis.

Delta Like Non-Canonical Notch Ligand (Dlk1) protein is a factor associated with several aspects of mammalian development, regeneration and disease, and is part of an imprinted gene network that controls tissue growth. Dlk1 is a paternally expressed imprinted gene (on mouse chromosome 12 and human chromosome 14) encoding an EGF membrane protein, which except for the lack of the DSL domain is similar in structure to the Delta/Notch family of proteins. A soluble form is generated by ectodomain cleavage, and both the membrane tethered—and the soluble form seem active.

Rodriguez et al. (Deletion of delta-like 1 homologue accelerates fibroblast-myofibroblast differentiation and induces myocardial fibrosis. EUROPEAN HEART JOURNAL, vol. 40, no. 12, 21 Mar. 2019, pages 967-978) discloses that deletion of Dlk1 accelerates fibroblast myofibroblast differentiation and induces myocardial fibrosis.

Xin-Quan et al. (Metabolism reprogramming: new insights of Dlk1 into cardiac fibrosis. EUROPEAN HEART JOURNAL, vol. 40, no. 43, 14 Nov. 2019, pages 3574-3574) and Lebeche Djamel (Response to ‘Metabolism reprogramming: new insights of Dlk1 into cardiac fibrosis. EUROPEAN HEART JOURNAL, vol. 40, no. 43, Nov. 14, 2019 (2019-11-14), pages 3575-3575) relate to insights of Dlk1 into cardiac fibrosis.

Tatiana V. et al. (Lack of periostin leads to suppression of Notchl signaling and calcific aortic valve disease. PHYSIOLOGICAL GENOMICS, vol. 39, no. 3, Nov. 1, 2009, pages 160-168) suggests that periostin is an inhibitor of Dlk1 expression.

Kaundal R-Lebeche D (Long term in vivo Dlk1 gene transfer protects against myocardial infarction-induced cardiac dysfunction. Circulation Research, Dec. 1, 2020, page e276) states that in vivo Dlk1 gene transfer protects against myocardial infarction-induced cardiac dysfunction.

Tatiana V. et al. (LTBP2 knockdown by siRNA reverses myocardial oxidative stress injury, fibrosis and remodelling during dilated cardiomyopathy. ACTA PHYSIOLOGICA, vol. 228, no. 3, Mar. 1, 2020) discloses the use of a siRNA targeting LTBP2 to treat dilated cardiomyopathy (DCM), in a rat model.

Bidur et al. (Cardiac Fibrosis in Proteotoxic Cardiac Disease is Dependent Upon Myofibroblast TGF-[beta] Signaling. JOURNAL OF THE AMERICAN HEART ASSOCIATION, vol. 7, no. 20, Oct. 16, 2018) discloses that inhibition of TGF-beta signaling in cardiac myofibroblasts reduces cardiac fibrosis.

WO 2010/107740 A2 relates to antisense oligonucleotides that modulate the expression of and/or function of Delta-like (1) homolog (DLK1), in particular, by targeting natural antisense polynucleotides of Delta-like (1) homolog (DLK1).

In conclusion, an improved protein inhibitor for preventing or alleviating complications following a heart condition would be advantageous, and in particular a more efficient and/or reliable inhibitor of a key protein associated with fibrotic scar tissue formation would be advantageous.

### SUMMARY OF THE INVENTION

An object of the present invention relates to inhibitors of specific proteins which otherwise leads to activation of for example transforming growth factor β (TGFβ) causing fibrotic scar tissue formation in a heart and/or around a heart. Inhibition of one such protein thus prevents and/or alleviates fibrotic scar tissue formation of a heart.

The inventors surprisingly found that Delta Like Non-Canonical Notch Ligand 1 (Dlk1) protein may control the concentration of the protein integrin beta-8 (Itgb8) which is an activator of TGFβ. It was also found that expression in adulthood of Dlk1 is restricted to the pericardium (peri-/epicardium), whereas Dlk1 expression in the myocardium is very low and almost absent in adult healthy subjects.

A protein inhibitor of the present invention seems to reduce TGFβ activity by inhibiting a protein directly or indirectly associated with activation of TGFβ. This could prevent and/or alleviate fibrotic scar tissue formation. Herein, a fibrotic scar tissue formation is for most embodiments considered to be a complication in the heart, preferably cardiac fibrosis. The cardiac fibrosis may have occurred in relation to acute trauma e.g. MI or alternatively by chronic conditions, such as a cancer of the heart, preferably mesothelioma or age-related changes.

A particular object of the present invention is therefore to provide inhibitors of Delta Like Non-Canonical Notch Ligand 1 (Dlk1) or integrin beta-8 (Itgb8) solving the above-mentioned problems of the prior art, wherein fibrotic scar tissue formation stiffens the heart muscle and reduces cardiac function in subjects having suffered or suffering to an acute or chronic condition.

Thus, a first aspect of the present invention relates to an inhibitor of Delta Like Non-Canonical Notch Ligand 1 (Dlk1) or integrin beta-8 (Itgb8) for use in preventing and/or alleviating complications of a heart condition;

wherein said complications of the heart condition are fibrotic scar tissue formation in the heart and/or around the heart of a subject, such as cardiac fibrosis.

In a preferred embodiment, the inhibitor is selected from the group consisting of
an oligonucleotide targeting the DLK1 gene, the Itgb8 gene or transcripts thereof; and
an anti-DLK1 antibody or an anti-Itgb8 antibody.

The inhibitors may be administered alone or in combination with other pharmaceutically acceptable substances.

A second aspect of the present invention relates to a pharmaceutical composition for use as described herein, wherein the composition comprises one or more inhibitors as described herein and one or more pharmaceutical acceptable excipients and/or carriers.

Dlk1 and Itgb8 have been found to be present in the heart and particularly in the pericardium and pericardial fluid. Specific administration to the pericardium and/or pericardial fluid by direct injection or by targeted drug delivery is therefore considered the preferred routes of delivery.

Levels of Dlk1 and/or Itgb8 in the pericardial fluid or blood, such as serum and/or plasma, of a subject suffering to an acute or chronic heart condition may therefore also be used in a diagnostic method as an indication of the degree/severity of fibrotic heart disease and whether the subject is likely to benefit from treatment with an inhibitor of the present invention or a pharmaceutical composition of the present invention or another suitable drug.

Thus, a third aspect of the present invention relates to a method for determining a likely effect of a treatment to prevent and/or alleviate cardiac fibrosis, the method comprising:
determining the level of Dlk1 and/or integrin beta-8 (Itgb8) protein in a sample of pericardial fluid or blood, such as serum and/or plasma, which has been obtained from a subject who suffers from a heart condition;
comparing said determined Dlk1 and/or Itgb8 level to a corresponding reference level; and
determining that said subject will
   likely not benefit from treatment to prevent or alleviate cardiac fibrosis or complications thereof, such as an inhibitor of Dlk1 or Itgb8, if said level is lower than said reference level, or
   likely benefit from treatment to prevent or alleviate cardiac fibrosis or complications thereof, such as an inhibitor of Dlk1 or Itgb8, if said level is equal to or higher than said reference level.

### BRIEF DESCRIPTION OF THE FIGURES

FIG. 1A shows relative quantitative real time PCR of Dlk1 mRNA in human myocardial specimens during development and adulthood (days past fertilization (dpf), n=1-3). FIG. 1B shows, Dlk1 immunohistochemistry of human pericardium (n=12). Arrows and arrowheads point at Dlk1+ and Dlk1− mesothelial cells, respectively, while * marks vasculature with a Dlk1+ endothelium.

FIG. 2A shows a Wilcoxon matched-pairs signed rank test of soluble Dlk1 (sDlk1) measured in plasma and pericardial fluid from patients (n=127) having undergone heart valve or coronary artery surgery. FIG. 2B, shows a simple linear regression based on the same data as presented in FIG. 2A.

FIG. 3A shows mRNA expression level of full-length Dlk1 during mouse dlk1^{+/+} heart development as divided into sections of embryonic development stage (E10.5-E16.5) and postnatal day (P1-P98) development. Also, FIG. 3B shows mRNA expression level of soluble Dlk1 during mouse dlk1^{+/+} heart development.

FIG. 4 shows that Itgb8 is reduced in Dlk1−/− mouse hearts during cardiac development and with a substantial reduction at embryonic development, E16.5, at which point Dlk1 levels peak in Dlk1^{+/+} hearts (see FIG. 3).

FIG. 5A shows Dlk1^{+/+} and Dlk1−/− mouse epicardium derived cells stimulated with TGFβ or vehicle (control) to undergo fibroblast differentiation, which was verified by a morphology change and qRT-PCR. Dlk1 and Itgb8 was as expected reduced in Dlk1−/− EPDCs as compared to Dlk1^{+/+} EPDCs. Furthermore, FIG. 5B shows an accompanied lower expression of the cardiac fibroblast EMT markers aSMA and Procollagen I. For Procollagen I, the levels were normalized to account for biological variations between experiments.

FIG. 6 shows that Itgb8 mRNA expression in Dlk1^{+/+} EPDCs treated with Itgb8 siRNA or control siRNA (SIRNA scramble) was knocked down by 75-90%. In line with this the procollagen I expression was lowered by 30-37%, a feature exaggerated in the presence of TGFβ.

FIG. 7A shows that Dlk1 mRNA expression increases after myocardial infarction (MI) is introduced in 10-weeks old female C57bl/6 mice by ligation of the left anterior descending (LAD) or sham surgery. Hearts were analysed at different time points by normalized relative quantitative RT-PCR for total Dlk1 mRNA.

FIG. 7B shows similarly obtained results for soluble Dlk1 mRNA, whereas

FIG. 7C shows ELISA results for circulating Dlk1 concentrations.

FIG. 8A shows scar size at different levels from apex to base using Massons' Trichrome histology, in hearts of 10 week old Dlk1^{+/+} and Dlk1^{-/-} mice that were introduced to myocardial infarction. The change in the area under the curve (AUC) shows that the scar size was lowered by 33.4% for hearts lacking Dlk1 expression (Dlk1^{-/-}) when compared to normal Dlk1^{+/+} control hearts. FIG. 8B shows scar size after MI at different levels from apex to base using Massons' Trichrome histology, in 10 week old mouse hearts overexpressing Dlk1 in cardiomyocytes (Dlk1^{fl/fl}×αMHC^{Cre/+ Tam}) as compared to normal hearts (Dlk1^{fl/fl}×αMHC^{Cre/- Tam}). The change in AUC shows that the scar size was increased by 21.6% for Dlk1 overexpressing hearts (Dlk1^{fl/fl}×αMHC^{Cre/+ Tam}) as compared to control hearts (Dlk1^{fl/fl}×αMHC^{Cre/- Tam}).

FIG. 9 shows scar size after MI at different levels from apex to base using Massons' Trichrome histology, in 10 week old mouse hearts overexpressing Dlk1 in peri-/epicardium derived cells such as fibroblasts (Dlk1^{fl/fl}×WT1^{GFPCre}) as compared to normal hearts (Dlk1^{fl/fl}×C57bl/6). The change in the area under the curve (AUC) shows that the scar size was increased by 24.8% for Dlk1 overexpressing hearts (Dlk1^{fl/fl}×WT1^{GFPCre}) as compared to control (Dlk1^{fl/fl}×C57bl/6).

FIG. 10 shows a scheme wherein the pericardial sac is dissected from adult Dlk1^{+/+} and Dlk1^{-/-} animals (n=4-6) at day 7 after ±pericardial sac lesion (PSL).

FIG. 11A shows Dlk1 and Itgb8 mRNA levels in pericardial sacs dissected from adult Dlk1^{+/+} and Dlk1^{-/-} animals (n=4-6) at day 7 after ±pericardial sac lesion (PSL Day 7) and analysed by qRT-PCR. In addition, FIG. 11B shows mRNA levels for the fibrotic markers Tcf21 and Procollagen I which are specifically induced by injury but reduced in pericardial specimens lacking Dlk1.

FIG. 12 shows mRNA levels of procollagen I in adult mouse interstitial cardiac ventricle fibroblasts after transfection with an extracellular cleavable Dlk1 gene (DLK1E-pLHCX-HA) and then stimulated with TGFβ or control and treated with vehicle, isotype-antibody (control) or anti-Dlk1-Ab. Inhibition of Dlk1 by anti-Dlk1-Ab decreases the procollagen I expression to non-TGFβ stimulated levels.

FIG. 13 shows siRNAs designed to decrease full length Dlk1 (Dlk1_{Total} SIRNA) or only soluble Dlk1 (Dlk1_{PS}SIRNA) in a mouse cell line, and their efficacy for Dlk1 knockdown as evaluated by measuring Dlk1 mRNA by qRT-PCR (13A and 13C), membrane bound/tethered Dlk1 (Dlk1^{M}) by Flow cytometry (13B) and soluble Dlk1 found by ELISA of the medium (13D). Use of the same siRNAs and their efficacy to knockdown Dlk1 in mouse peri-epicardial cells in culture as determined by qRT-PCR is shown in 13E.

FIG. 14 shows use of different siRNAs designed for efficient knockdown of Dlk1 mRNA in a culture of human cells and determined by qRT-PCR.

FIG. 15 shows the applicability of siRNA delivery to the pericardial compartment in mouse. A) Soluble Dlk1 protein-Serum; B) Dlk1 mRNA-Pericardium; and C) Itgb8 mRNA-Pericardium.

The present invention will now be described in more detail in the following.

### DETAILED DESCRIPTION OF THE INVENTION

### Definitions

Prior to discussing the present invention in further details, the following terms and conventions will first be defined:

### Inhibitor

In the present context, the term “inhibitor” refers to a chemical substance capable of decreasing or preventing a specific function and/or activity of a target gene or protein. For example, the activity of a protein may be decreased by reducing the expression of that protein.

### Dlk1

In the present context, the term “Dlk1” may depending on the context refer to a Delta Like Non-Canonical Notch Ligand (Dlk1) gene, or mRNA expressed by Dlk1, or a corresponding Dlk1 protein. Human and mouse Dlk1 are categorized with the UniProtKB numbers P80370 and Q09163, respectively.

### Itgb8

In the present context, the term “Itgb8” may depending on the context refer to an integrin beta-8 (Itgb8) gene, or mRNA expressed by Itgb8, or a corresponding Itgb8 protein. Human and mouse Itgb8 are categorized with the UniProtKB numbers P26012 and Q0VBD0, respectively. Herein, Itgb8 may also be written as Itgβ8.

### Heart Condition

In the present context, the term “heart condition” refers to a condition or disease in the heart, which negatively affects the heart's ability to work as normal.

### Acute

In the present context, the term “acute” should be understood as something that is triggered by a specific event, such as triggered by a surgical procedure, heart attack or similar. However, the term is not restricted only to situations that need to be acted upon immediately, though such situations are definitely covered by the term.

### Fibrotic Scar Tissue Formation

In the present context, the term “fibrotic scar tissue formation” refers to a condition wherein a subject or organ produces an excessive amount of fibrotic scar tissue. The abnormal formation of scar tissue is controlled by accumulation of extracellular matrix components, such as collagen. Too much scar tissue may decrease function of an organ, such as the heart, whereby the heart is unable to maintain a normal blood circulation. The fibrotic scar tissue formation may be activated by injury, aging, and/or disease.

### Cardiac Fibrosis

In the present context, the term “cardiac fibrosis” refers to fibrotic scar tissue formation in the heart or around the heart. The formed fibrotic scars may, among other cardiac dysfunctions, result in a stiffened myocardial matrix whereby heart function is severely reduced. Another implication of cardiac fibrosis is an abnormal thickening of the heart valves due to excessive proliferation of cardiac fibroblasts, possibly leading to valvular dysfunction and heart failure. Similarly, pericardial adhesions is a common finding after heart surgery-induced fibrosis. Moreover, pericarditis and associated malignancies embrace TGFβ signalling, the major fibrotic stimulator and are both life threatening.

### Myocardial Infarction

In the present context, the term “myocardial infarction” refers to a heart attack which is a condition wherein blood flow of at least one of the hearts coronary arteries is blocked or lower than normal. Patients suffering from atherosclerosis are in particularly risk of developing a heart attack.

### Myocardial Remodelling (REM)

In the present context, the term “myocardial remodelling” refers to a process wherein molecular, cellular and interstitial changes leads to changes in size, mass, geometry and function of the myocardium. Often, the consequences is a gradual cardiac enlargement and/or cardiac dysfunction.

### Pericardium

In the present context, the term “pericardium” refers to the pericardial sac enclosing the heart. The sac comprises an outer fibrous pericardium layer and an inner serous pericardium layer (“the epicardium”). Between the two layers is the pericardial cavity.

### Pericardial Fluid

In the present context, the term “pericardial fluid” refers to the fluid in the pericardial cavity.

### Intracardiac Administration

In the present context, the term “intracardiac administration” (I-CARDI) refers to administration into the heart muscle or circulatory system.

### Intrapericardial Fluid Administration

In the present context, the term “intrapericardial fluid administration” refers to an intrapericardial administration (I-PERICARD) which is an administration within the pericardium, preferably to the intrapericardial fluid.

### Oligonucleotide

In the present context, the term “oligonucleotide” refers to a sequence of DNA or RNA nucleotide residues comprising nucleobases. Oligonucleotides can bind their complementary base sequences to form duplexes (double-stranded fragments) or even fragments of a higher order. They may be on a linear form or exist as circular oligonucleotide molecules. When referring to the length of a sequence, reference is usually made to the number of nucleotide units or to the number of nucleobases. An oligonucleotide used as an inhibitor is a molecule capable of inhibiting the function of a target. Oligonucleotide inhibitors may be selected from the non-limiting list of antimiRs, BlockmiRs, antisense oligonucleotides, oligonucleotide decoys, oligonucleotide sponges, siRNA's, and circular oligonucleotides. The oligonucleotides may be modified in order to increase stability and efficacy.

### Antibody

In the present context, the term “antibody” refers to a protein that may specifically bind to a corresponding antigen. Antibodies may particularly stem from the immune system of e.g. mammals, and may be directed towards antigens related to foreign bodies. An antibody is an intact immunoglobulin having two light (L) and two heavy (H) chains inter-connected by disulfide bonds. Each heavy chain (about 50-70 kDa) is comprised of a heavy chain variable domain (VH) and a heavy chain constant region (CH). Each light chain (about 25 kDa) is composed of a light chain variable domain (VL) and a light chain constant region (CL). The VH and VL domains can be subdivided further into regions of hypervariability, termed “complementarity determining regions” (CDRs), interspersed with regions that are more conserved, termed “framework regions” (FRs).

Thus, a single isolated antibody or fragment hereof e.g. Fab′ may be originating from the non-limiting list of a polyclonal antibody, a monoclonal antibody, a synthetic antibody, a recombinant antibody, a chimeric antibody, a heterochimeric antibody, or a humanized antibody. The term antibody is used both to refer to a homogeneous molecular mixture, or a mixture such as a serum product made up of a plurality of different molecular entities.

asRNA

In the present context, the term “asRNA” refers to antisense RNA which is a single stranded RNA capable of hybridizing complementary messenger RNA (mRNA) and thereby inhibit or block translation of a specific protein based on that type of mRNA.

siRNA

In the present context, the term “siRNA” refers to small interfering RNA, also called silencing RNA or short interfering RNA. An siRNA is a double-stranded and non-coding RNA molecule that disrupts or degrades mRNA having a complementary nucleotide sequence. Thus, siRNA may be used for decreasing or blocking expression of a gene transcribing a target mRNA.

### Pharmaceutical Acceptable

In the present context, the term “pharmaceutically acceptable” refers to molecular entities, compositions and methods that are suitable for use with humans and/or animals without undue adverse side effects (such as toxicity, irritation and allergic response) commensurate with a reasonable benefit/risk ratio.

### Excipient

In the present context, the term “excipient” refers to a natural or synthetic substance formulated alongside the active or therapeutic ingredient (an ingredient that is not the active ingredient) of a medication, included for the purpose of stabilization, bulking, or to confer a therapeutic enhancement on the active ingredient in the final dosage form, such as facilitating drug absorption, reducing viscosity, enhancing solubility, adjusting tonicity, mitigating injection site discomfort, depressing the freezing point, or enhancing stability. The term may refer to a diluent, adjuvant, carrier, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

### Carrier

In the present context, the term “carrier” refers to any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Inhibitor of Dlk1 or Itgb8 for Use in Preventing and/or Alleviating Complications of a Heart Condition

Inhibitors of Dlk1 or Itgb8 for use in preventing or alleviating fibrotic scar tissue formation in or around a heart of a patient having suffered or who is still suffering to an acute or chronic heart condition are provided.

Transforming growth factor β is a mediator of fibrotic scar tissue formation when activated, e.g. by Itgb8. In addition, it was a great surprise to the inventors of the present invention to find that levels of Itgb8 seem to be controlled by the expression and presence of Dlk1. It is however possible that Dlk1 in addition to this mechanism also affects collagen accumulation and fibrosis through another mechanism.

Administration of inhibitors for inhibition of Dlk1 or Itgb8 is therefore an alternative or improved means for preventing and/or alleviating fibrotic scar tissue formation in or around the heart.

A first aspect of the present invention relates to an inhibitor of Delta Like Non-Canonical Notch Ligand 1 (Dlk1) or integrin beta-8 (Itgb8) for use in preventing and/or alleviating complications of a heart condition;

wherein said complications of the heart condition are fibrotic scar tissue formation in the heart and/or around the heart of a subject, such as cardiac fibrosis.

In a preferred embodiment, the inhibitor is selected from the group consisting of
an oligonucleotide targeting the DLK1 gene, the Itgb8 gene or transcripts thereof; and
an anti-DLK1 antibody or an anti-Itgb8 antibody.

Another embodiment of the present invention relates to the inhibitor for use, wherein the heart condition is an acute condition or a chronic condition. However, a more specific embodiment of the present invention relates to the inhibitor for use, wherein the heart condition is selected from the group consisting of myocardial infarction, aortastenose, heart surgery, age-related heart disorders, and cancer of the heart.

In one preferred embodiment, the heart condition is myocardial infarction.

In another preferred embodiment, the heart condition is heart surgery.

Expression of Dlk1, and thus the levels of Dlk1 and Itgb8, seems to increase in response to myocardial infarction and heart surgery, as may be derived from Example 6 and Example 7. One embodiment of the present invention thus relates to the inhibitor for use, wherein the heart condition is an acute heart condition, such as myocardial infarction or heart surgery.

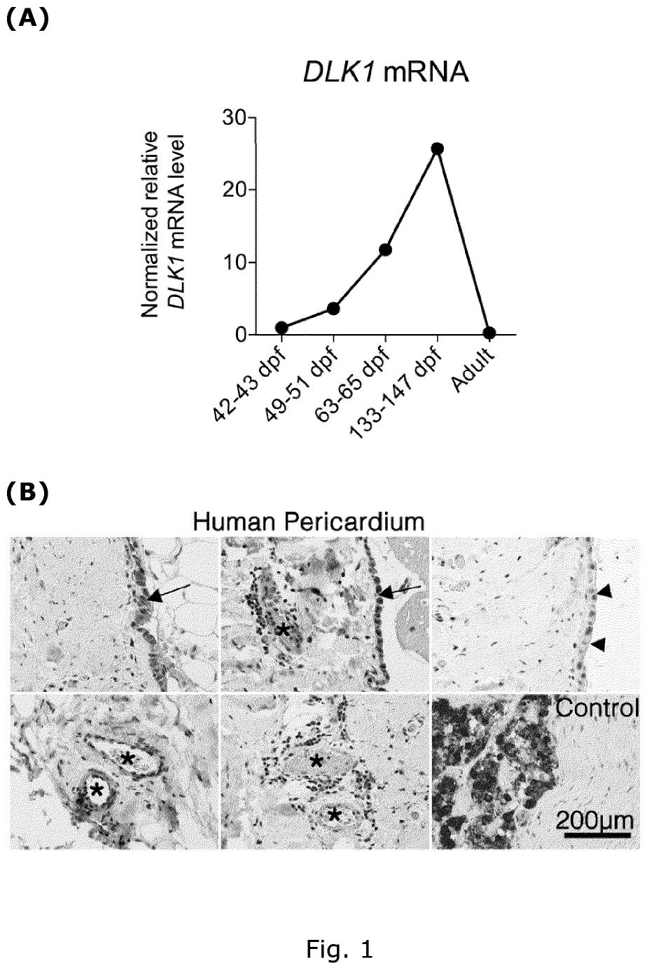

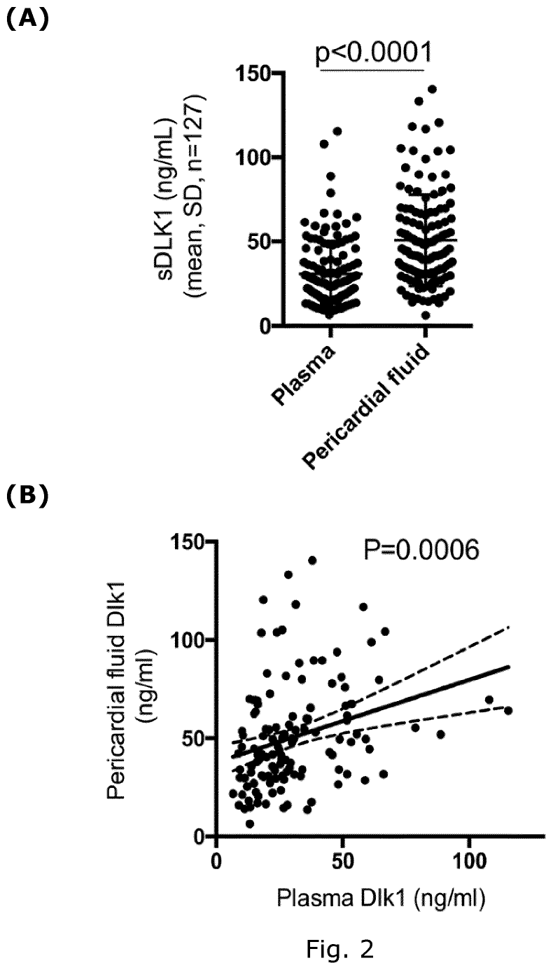

Additionally, Dlk1 is mainly in the pericardium in a membrane tethered form or in the pericardial fluid in a soluble form, but both forms seem to be active. Example 3 describes that Dlk1+ cells are more or less absent in the myocardium of human patients (FIG. 1A), while a substantial and consistent Dlk1 expression is present in the pericardium (FIG. 1B). Analysis results of pericardial fluids (PF) and venous plasmas from 127 patients having heart conditions is described in Example 3, and shows that soluble Dlk1 is secreted into the pericardial fluid (FIG. 2A). Moreover, the mean sDlk1 ratio was found to be twice as high in the pericardial fluid than in the venous plasma and with a linear correlation between the two (FIG. 2B).

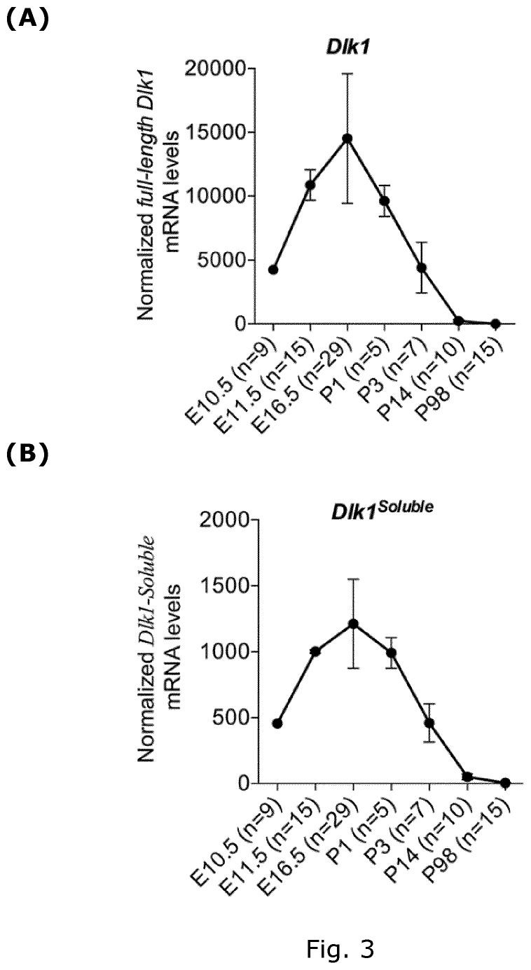

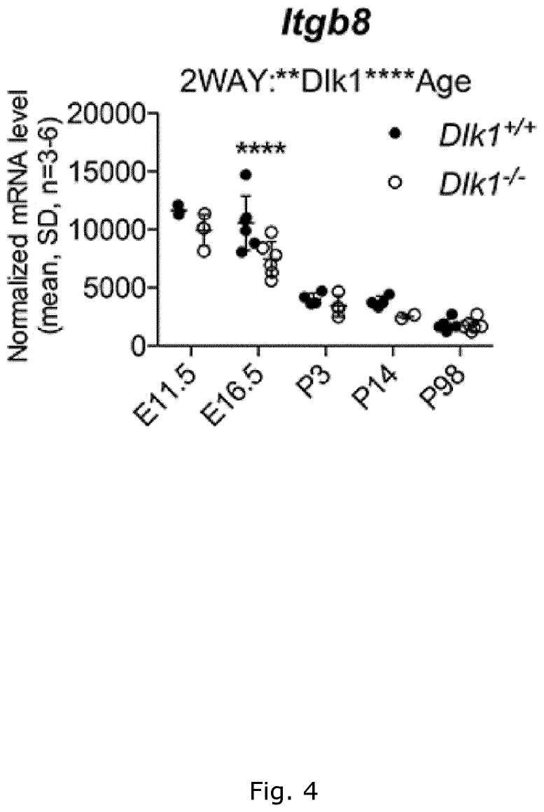

An increased expression of Dlk1 may lead to increased expression of Itgb8 as Example 5 shows for epicardium derived cells obtained from Dlk1^{+/+} and Dlk1^{-/-} mice. The level of Itgb8 could increase in Dlk1^{+/+} species during cardiac development, with a substantial increase at the same time as the Dlk1 level is peaking (i.e. at embryonic development stage E16.5) (compare FIG. 4 with FIG. 3A and FIG. 3B).

The pericardial and pericardial fluids are considered to be particularly relevant compartments for delivery of the inhibitors, either for inhibition of the soluble or tethered form of the target protein. Many different routes of delivery may be relevant, such as but not limited to, direct injection, targeted injection, target specific system delivery. Thus, an embodiment of the present invention relates to the inhibitor for use, wherein the inhibitor is delivered to the pericardium and/or pericardial fluid of the heart.

Any heart condition directly or indirectly leading to increased fibrotic scar tissue formation in and/or around a heart is relevant for the purpose of preventing or alleviating heart complications using the inhibitors as described herein. For some complications the increased fibrotic scar tissue formation may be on the surface of the heart, such as in and around the pericardial sac and its two membranes (peri- and epicardium), or the scars may be formed in tissues in close proximity to the surface of the heart, e.g. surface of the pericardial sac, whereby the stiff and rough scar tissues increases wear and tear on the working heart.

Other complications are caused by increased fibrotic scar tissue formation with deposition of extracellular matrix in the heart, thereby stiffening the heart and potentially leading to a life-threatening situation. Cardiac fibrosis is one such complication and a known cause of decreased function of the cardiac muscle due to a reduced flexibility thereof, while also thickening the valves of the heart to an extend where the valvular system may fail. A particularly important embodiment of the present invention therefore relates to the inhibitor for use, wherein the heart condition may cause or causes cardiac fibrosis.

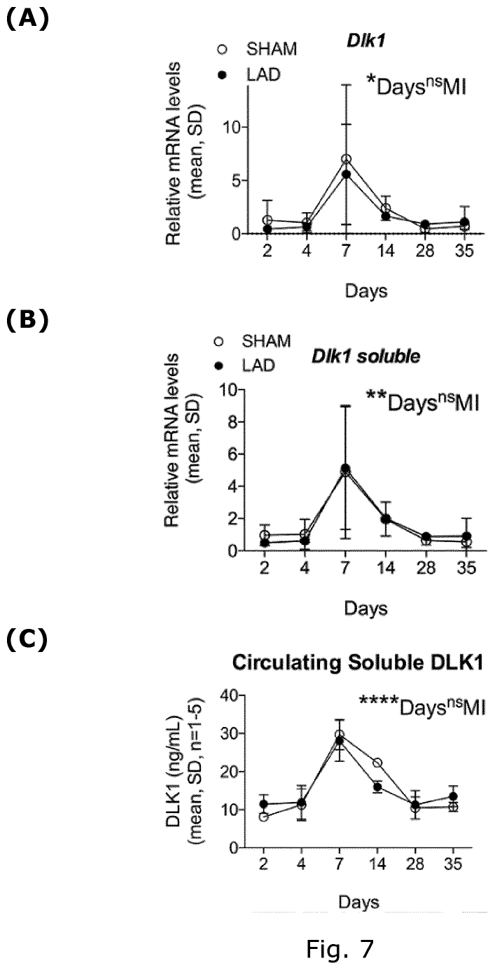

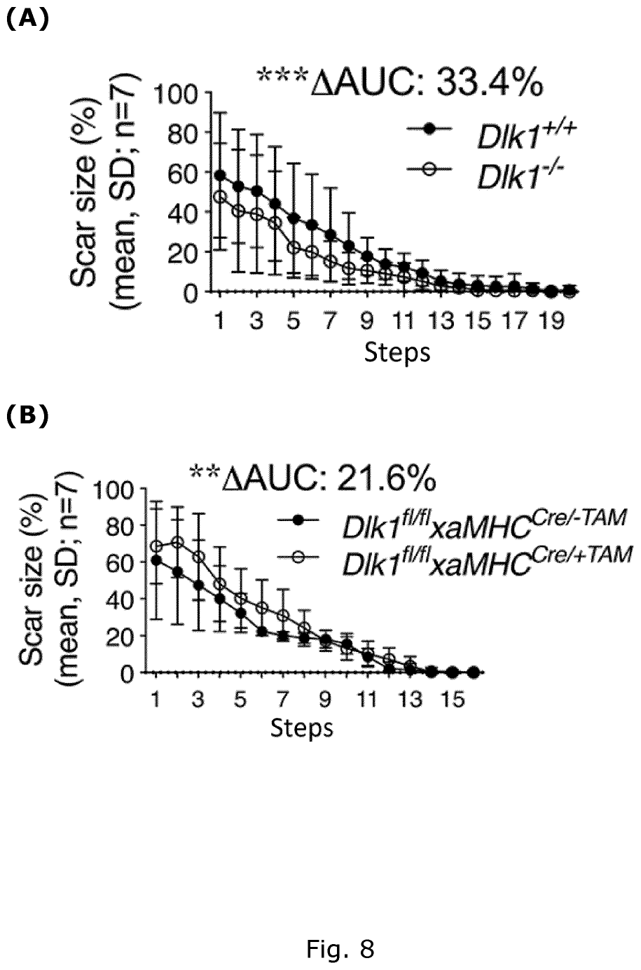

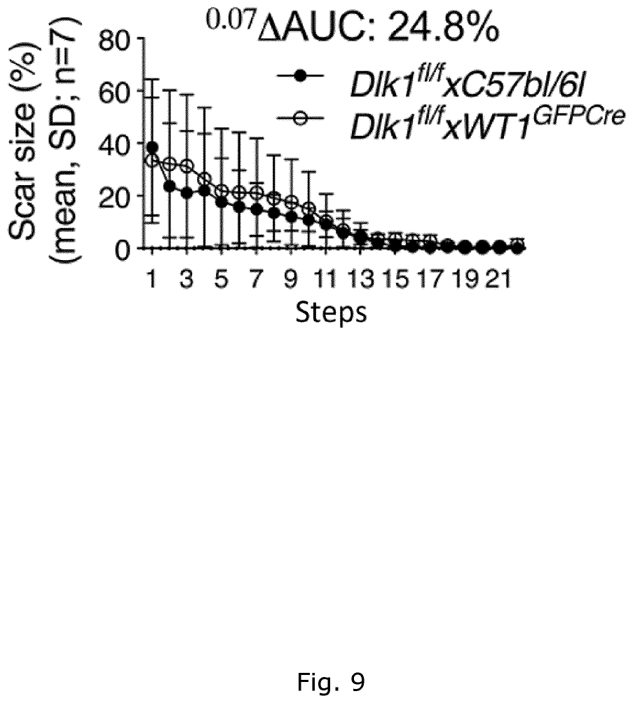

The heart condition directly or indirectly leading to increased fibrotic scar tissue formation in and/or around the heart may be an acute or a chronic condition and may relate but is not limited to myocardial infarction, aortastenose, heart surgery, or to a cancer of the heart. Example 6, describes how surgery with opening and tearing of the pericardial sac in mice increases expression of Dlk1, which peaks seven days after the surgical procedure. Seven days after surgery the level of tethered Dlk1 peaks (FIG. 7A) together with the level of soluble Dlk1 (FIG. 7B, FIG. 7C). In addition, 8 weeks after introduction of myocardial infarction scar size was decreased by 33.4% in mice deficient of the Dlk1 gene as compared to mice possessing the gene (FIG. 8A). On the opposite, scar size is increased with 21.6% (FIG. 8B) and 24.8% (FIG. 9) in two different sets of transgenic mice overexpressing Dlk1 specifically in the heart.

The inhibitor or pharmaceutical composition comprising the inhibitor may be administered to the subject before, during or after a surgical procedure inflicting an increased fibrotic scar tissue formation. Thus, an embodiment of the present invention relates to the inhibitor for use, wherein the subject has undergone heart surgery or is undergoing heart surgery.

However, the heart condition may also be a chronic disease inflicting an increased fibrotic scar tissue formation. Cancers are considered particularly relevant in this regard and an embodiment of the present invention therefore relates to the inhibitor for use, wherein the subject suffers from cancer of the heart, such as cancer in the pericardium, preferably mesothelioma.

The inhibitor or pharmaceutical composition comprising the inhibitor may be administered to any subject in need thereof, particularly humans. However, in its broadest interpretation the subject may be any kind of mammal, whereby pets and household animals are included. Adult specimen are considered more relevant subject to treatment with the inhibitor, because activity of Dlk1 seems important during the development stage of the young mammal as the protein increases the amount Itgb8, which further activates TGFβ that is crucial for growth and development of the young body. Example 3 describes how Dlk1 levels increase during human heart development and then declines to be become nearly abolished in normal adult ventricles as shown in FIG. 1A. Expression of Dlk1 hereafter is then mainly restricted to the pericardium and may be further induced in subjects subjected to heart surgery and eventually also heart disease. Thus, an embodiment of the present invention relates to the inhibitor for use, wherein the subject is a mammal, such as a human, preferably an adult human, such as an adult human of an age above 18.

The fibrotic scar tissue formation is controlled by activation of TGFβ which depends on the level of Itgb8, which is controlled by expression of Dlk1. Because the overall goal is to avoid or decrease activation of TGFβ, the inhibitor may be an inhibitor of Dlk1 or Itgb8. One embodiment of the present invention therefore relates to the inhibitor for use, wherein the inhibitor inhibits Dlk1. Yet, another embodiment of the present invention relates to the inhibitor for use, wherein the inhibitor inhibits Itgb8.

The inhibitor may be any type of inhibitor, such as a small molecule, oligonucleotide or antibody. A particular embodiment of the present invention thus relates to the inhibitor for use, wherein the inhibitor is selected from the group consisting of an oligonucleotide, an antibody or a small molecule.

In the present context, a small molecule may be, for example, a guide RNA for CRISPR/Cas9 mediated gene knockout.

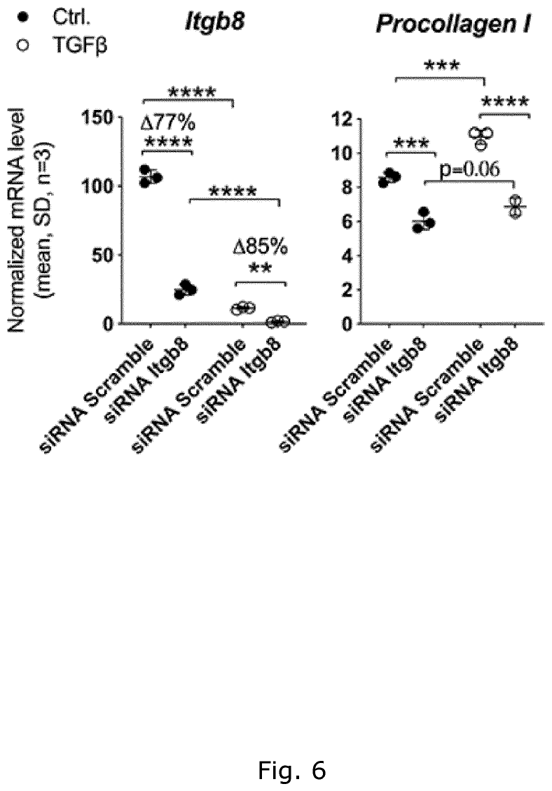

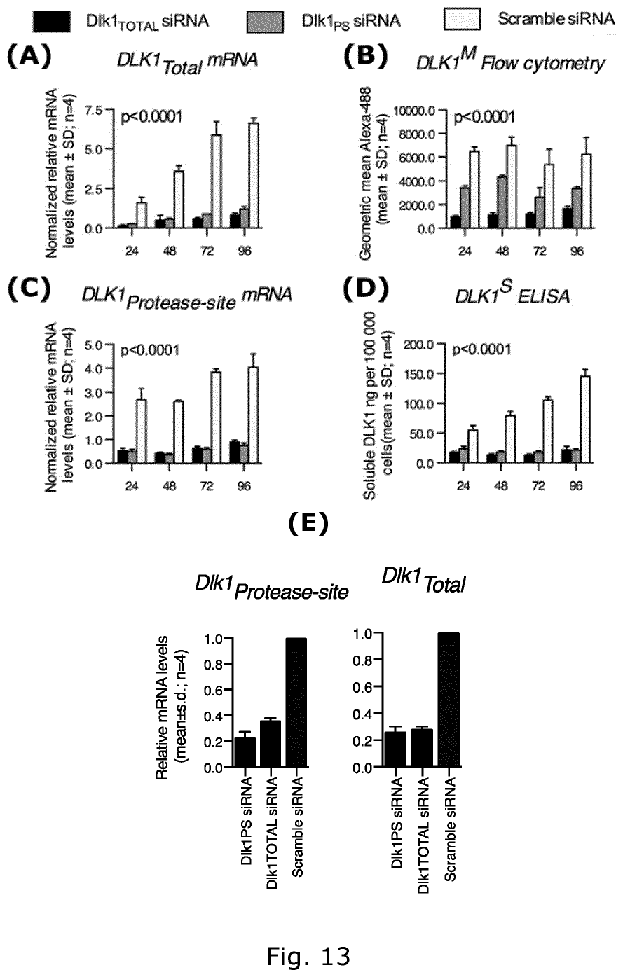

A protein may be inhibited in several distinct ways, such as by an inhibitor that prevents efficient production of the protein and thereby lowers the amount (i.e. level) of that protein. Thus, an embodiment of the present invention relates to the inhibitor for use, wherein the inhibitor is an oligonucleotide that inhibits gene expression of Dlk1 protein and/or Itgb8 protein. In other words, an embodiment of the present invention relates to the inhibitor for use, wherein the inhibitor is an oligonucleotide that inhibits translation of mRNA transcribed from a Dlk1 gene sequence and/or Itgb8 gene sequence. As shown in Example 9 and FIG. 13(A-D) and FIG. 6 oligonucleotides (siRNA) are able to inhibit Dlk1 and Itgb8, respectively.

In an embodiment, the oligonucleotide is complementary to the DLK1 gene, the Itgb8 gene or transcripts thereof.

However, a preferred embodiment of the present invention relates to the inhibitor for use, wherein the oligonucleotide is a single stranded oligonucleotide or double stranded oligonucleotide. Whereas, a particularly preferred embodiment of the present invention relates to the inhibitor for use, wherein the oligonucleotide is selected from the group consisting of an antisense RNA (asRNA), antisense DNA (asDNA), guide RNA (gRNA), and small interfering RNA (siRNA). An even more specific embodiment of the present invention relates to the inhibitor for use, wherein the oligonucleotide is selected from the group consisting of SEQ ID NO: 1, 2, 3, 4, 5, 6, 7, 8, 9, 10, 11, 12, 13, 14, 15, 16, 17, 18, 19, 20, 21, 22. The sequences are listed in Table 1.

The following embodiments relates to Dlk1 Homo sapiens delta like non-canonical Notch ligand 1 (Dlk1, NCBI Reference Sequence: NM_003836.7). One embodiment of the present invention relates to the inhibitor for use, wherein the oligonucleotide comprises a sequence of SEQ ID NO: 1 or 2, that are related to a position in Dlk1 (sense) at bp 263-287 (in CDS). Another embodiment of the present invention relates to the inhibitor for use, wherein the oligonucleotide comprises a sequence selected from the group consisting of SEQ ID NO: 3, 4, 5, 6, 7, 8, 9, and 10, that are related to a position in Dlk1 at bp 1270-1610 (i.e. in 3′+3′UTR-exon 6). Yet another embodiment of the present invention relates to the inhibitor for use, wherein the oligonucleotide comprises a sequence of SEQ ID NO: 11 or 12, that are related to a position of exon 5 in Dlk1. A further embodiment of the present invention relates to the inhibitor for use, wherein the oligonucleotide comprises a sequence of SEQ ID NO: 13 or 14, that are related to a position of exon 2 in Dlk1. A specific embodiment of the present invention relates to the inhibitor for use, wherein the oligonucleotide comprises a sequence of SEQ ID NO: 15 or 16, that are related to a position of exon 5 in Dlk1. A specific embodiment of the present invention relates to the inhibitor for use, wherein the oligonucleotide comprises a sequence selected from the group consisting of SEQ ID NO: 17, 18, 19, 20, 21, and 22, that are related to a position of exon 6 in Dlk1.

TABLE 1
RNA/DNA for inhibition of human Dlk1 and/or Itgb8.

| SEQ ID NO: | Sequence | Strand |
|---|---|---|
| 1 | 5′ CCUGCAACCCCCAAAAUGGAUUCTG 3′ | + |
| 2 | 5′ CAGAAUCCAUUUUGGGGGUUGCAGGCC 3′ | − |
| 3 | 5′ CUCUCUUAAUGCAUGAUACAGAATA 3′ | + |
| 4 | 5′ UAUUCUGUAUCAUGCAUUAAGAGAGAG 3′ | − |
| 5 | 5′ CUUGUGUCAAAUCUGGUGAACGCTA 3′ | + |
| 6 | 5′ UAGCGUUCACCAGAUUUGACACAAGAG 3′ | − |
| 7 | 5′ CUCUCUCUUAAUGCAUGAUACAGAA 3′ | + |
| 8 | 5′ UUCUGUAUCAUGCAUUAAGAGAGAGAA 3′ | − |
| 9 | 5′ AAAUAAGUAUGUUAUUCUAAAAUCT 3′ | + |
| 10 | 5′ AGAUUUUAGAAUAACAUACUUAUUUCU 3′ | − |
| 11 | 5′ CUCUUAAUGCAUGAUACAGAAUAAT 3′ | + |
| 12 | 5′ AUUAUUCUGUAUCAUGCAUUAAGAGAG 3′ | − |
| 13 | 5′ AAAAUGGAUUCUGCGAGGAUGACAA 3′ | + |
| 14 | 5′ UUGUCAUCCUCGCAGAAUCCAUUUUGG 3′ | − |
| 15 | 5′ GGCUUCUCAGGCAAUUUCUGCGAGA 3′ | + |
| 16 | 5′ UCUCGCAGAAAUUGCCUGAGAAGCCAG 3′ | − |
| 17 | 5′ CUUAAUGCAUGAUACAGAATT 3′ | + |
| 18 | 5′ UUCUGUAUCAUGCAUUAAGAG 3′ | − |
| 19 | 5′ CAAACGCAAUGCAAAAACATT 3′ | + |
| 20 | 5′ UGUUUUUGCAUUGCGUUUGTC 3′ | − |
| 21 | 5′ GCUUACAUAUAUUGUCUUUTT 3′ | + |
| 22 | 5′ AAAGACAAUAUAUGUAAGCGT 3′ | − |

Target sequence: Homo sapiens delta like non-canonical Notch ligand 1 (Dlk1) of NCBI Reference Sequence: NM_003836.7. Bases with underline refers to RNA whereas bases in bold phase refers to DNA.

A particular embodiment of the present invention relates to the inhibitor for use, wherein the oligonucleotide is a double stranded RNA comprising SEQ ID NO: 1 and SEQ ID NO: 2. Another embodiment of the present invention relates to the inhibitor for use, wherein the oligonucleotide is a double stranded RNA comprising SEQ ID NO: 3 and SEQ ID NO: 4. A further embodiment of the present invention relates to the inhibitor for use, wherein the oligonucleotide is a double stranded RNA comprising SEQ ID NO: 5 and SEQ ID NO: 6. Yet another embodiment of the present invention relates to the inhibitor for use, wherein the oligonucleotide is a double stranded RNA comprising SEQ ID NO: 7 and SEQ ID NO: 8. One more embodiment of the present invention relates to the inhibitor for use, wherein the oligonucleotide is a double stranded RNA comprising SEQ ID NO: 9 and SEQ ID NO: 10. An additional embodiment of the present invention relates to the inhibitor for use, wherein the oligonucleotide is a double stranded RNA comprising SEQ ID NO: 11 and SEQ ID NO: 12. A special embodiment of the present invention relates to the inhibitor for use, wherein the oligonucleotide is a double stranded RNA comprising SEQ ID NO: 13 and SEQ ID NO: 14. Still another embodiment of the present invention relates to the inhibitor for use, wherein the oligonucleotide is a double stranded RNA comprising SEQ ID NO: 15 and SEQ ID NO: 16. A particular embodiment of the present invention relates to the inhibitor for use, wherein the oligonucleotide is a double stranded RNA comprising SEQ ID NO: 17 and SEQ ID NO: 18. A particular embodiment of the present invention relates to the inhibitor for use, wherein the oligonucleotide is a double stranded RNA comprising SEQ ID NO: 19 and SEQ ID NO: 20. A particular embodiment of the present invention relates to the inhibitor for use, wherein the oligonucleotide is a double stranded RNA comprising SEQ ID NO: 21 and SEQ ID NO: 22.

An embodiment of the present invention relates to the inhibitor for use, wherein the length of the oligonucleotide is 18 to 30 base pairs, such as 18-29, such as 20-28, such as 23-28, such as 24-28, preferably 25-27 base pairs.

Other inhibitors may decrease or completely destroy a specific function of the target protein. This type of inhibitor is considered to be a small molecule or antibody. A first alternative embodiment of the present invention therefore relates to the inhibitor for use, wherein the inhibitor is a small molecule that inhibits Dlk1 protein and/or Itgb8 protein mediated activation of transforming growth factor β (TGFβ).

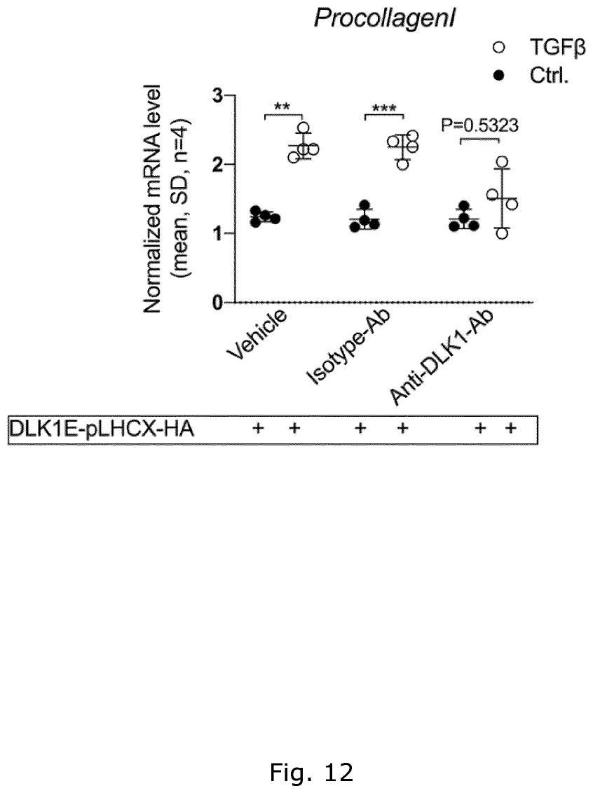

As shown in FIG. 12, targeting of Dlk1 with an anti-Dlk1-ab delimits production of procollagen I (a key factor in TGFβ induced fibrotic scar tissue formation) even when the cells are TGFβ stimulated. Thus, a second alternative embodiment of the present invention relates to the inhibitor for use, wherein the antibody is selected from the group consisting of a polyclonal antibody, a monoclonal antibody, an antibody, wherein the heavy chain and the light chain are connected by a flexible linker, an Fv molecule, an antigen binding fragment, a Fab fragment, a Fab′ fragment, a F(ab′)2 molecule, a fully human antibody, a humanized antibody, a nanobody and a chimeric antibody.

Again, in a preferred embodiment, the antibody is an anti-DLK1 antibody or an anti-Itgb8 antibody.

In another embodiment, the antibody binds directly to DLK1 and/or Itgb8.

In yet another embodiment, the antibody is a neutralizing antibody.

The inhibitor or pharmaceutical composition comprising the inhibitor may be administered to the subject by any pharmaceutically acceptable route. However, the administration route considered most relevant is a direct delivery to the specific target areas. Thus, a preferred embodiment of the present invention relates to the inhibitor for use, wherein the inhibitor is administered by intracardiac or intrapericardial fluid administration.

One embodiment of the present invention relates to the inhibitor for use, wherein the inhibitor is administered within a week of a myocardial infarction. Furthermore, an embodiment of the present invention relates to the inhibitor for use, wherein the inhibitor is administered within 72 hours of a myocardial infarction, such as within 48 hours, such as 36 hours, preferably within 24 hours, more preferably within 12 hours, more preferably with 6 hours, within 2 or within 1 hour of myocardial infarction.

Another embodiment of the present invention relates to the inhibitor for use, wherein the inhibitor is administered within a week from the beginning of a heart surgery. Furthermore, an even further embodiment of the present invention relates to the inhibitor for use, wherein the inhibitor is administered within 72 hours from the beginning of a heart surgery, such as within 48 hours, such as 36 hours, preferably within 24 hours, more preferably within 12 hours, such as during heart surgery.

Heart surgery is in the present context surgery performed on the heart, however the beginning of heart surgery is the time of the first incision, preferably the first incision of the skin, more preferably the first incision of the heart.

Further advanced embodiments of the present invention, includes an embodiment relating to the inhibitor for use, wherein Dlk1 is present in the pericardium and/or pericardial fluid. Another advanced embodiment of the present invention relates to the inhibitor for use, wherein inhibition of Dlk1 reduces myocardial remodelling, whereby cardiac fibrosis is prevented or alleviated. A particularly advanced embodiment of the present invention relates to the inhibitor for use, wherein inhibition of Dlk1 reduces epithelial to mesenchymal transition (EMT) of epicardium derived cells (EPDC) to form cardiac fibroblast or EMT of cardiac fibroblasts to form myofibroblasts, whereby cardiac fibrosis is prevented and/or alleviated. Yet, another embodiment of the present invention relates to the inhibitor for use, wherein inhibition of Dlk1 leads to a reduction of Itgb8 in the subject, whereby cardiac fibrosis is prevented and/or alleviated.

The inhibitor may be administered to the subject alone or in a pharmaceutical composition comprising the inhibitor(s).

Thus, a second aspect of the present invention relates to a pharmaceutical composition for use as described herein, wherein the composition comprises one or more inhibitors as described herein and one or more pharmaceutical acceptable excipients and/or carriers.

Any uses of the inhibitor as described herein also applies to the pharmaceutical composition as described herein.

The Dlk1 and Itgb8 proteins may be present in the pericardial fluid as a result of a condition and/or complication of the heart. Thus, measuring the concentration of specific proteins in the pericardial fluid obtained from a subject may be used to decide the severity of the disease or condition and to determine whether the subject would benefit from treatment with an inhibitor of the present invention.

### Diagnostic Method

A third aspect of the present invention relates to a method for determining a likely effect of a treatment to prevent and/or alleviate cardiac fibrosis, the method comprising:
determining the level of Dlk1 and/or integrin beta-8 (Itgb8) protein in a sample of pericardial fluid or blood, such as serum and/or plasma, which has been obtained from a subject who suffers from a heart condition;
comparing said determined Dlk1 and/or Itgb8 level to a corresponding reference level; and
determining that said subject will
   likely not benefit from treatment to prevent or alleviate cardiac fibrosis or complications thereof, such as an inhibitor of Dlk1 or Itgb8, if said level is lower than said reference level, or
   likely benefit from treatment to prevent or alleviate cardiac fibrosis or complications thereof, such as an inhibitor of Dlk1 or Itgb8, if said level is equal to or higher than said reference level.

One embodiment of the present invention relates to the method, wherein said determination of the level of Dlk1 and/or Itgb8 is performed using a method selected from the group consisting of immunohistochemistry, immunocytochemistry, flow cytometry, ImageStream, Western Blotting, qPCR, RT-PCR, qRT-PCR, Olink, ELISA, Luminex, Multiplex, Immunoblotting, TRF-assays, immunochromatographic lateral flow assays, Enzyme Multiplied Immunoassay Techniques, RAST test, Radioimmunoassays (RIA), immunofluorescence and immunological dry stick assays.

A preferred embodiment of the present invention relates to the method, wherein said method is an antibody based assay selected from the group consisting of ELISA, Luminex, and radioimmunoassay (RIA).

An embodiment of the present invention relates to the method, wherein said heart condition is selected from the group consisting of myocardial infarction, aortastenose, heart surgery, age-related heart disorders, and cancer of the heart.

An embodiment of the present invention relates to the method, wherein the sample has been obtained from said subject within a week of myocardial infarction. More specifically, an embodiment of the present invention relates to the method, wherein the sample has been obtained from said subject within 72 hours of a myocardial infarction, such as within 48 hours, such as 36 hours, preferably within 24 hours, more preferably within 12 hours, more preferably with 6 hours, within 2 or within 1 hour of myocardial infarction.

An embodiment of the present invention relates to the method, wherein the sample has been obtained from said subject within a week of heart surgery. More specifically, an embodiment of the present invention relates to the method, wherein the sample has been obtained from said subject within 72 hours from the beginning of a heart surgery, such as within 48 hours, such as 36 hours, preferably within 24 hours, more preferably within 12 hours, such as during heart surgery.

Yet an aspect of the present invention relates to a method for determining if a subject is at risk of suffering from cardiac fibrosis, the method comprising
determining the level of Dlk1 and/or integrin beta-8 (Itgb8) protein in a sample of pericardial fluid or blood, such as serum and/or plasma, which has been obtained from a subject;
comparing said determined Dlk1 and/or Itgb8 level to a corresponding reference level; and
determining that said subject
   likely suffers from cardiac fibrosis or complications thereof, if said level is equal to or higher than said reference level; or
   likely not suffering from cardiac fibrosis or complications thereof, if said level is lower than said reference level.

As shown e.g. in example 3, levels of Dlk1 and/or integrin beta-8 (Itgb8) protein may be measured in pericardial fluid and/or blood, such as serum and/or plasma in humans. Since, in examples 5-8 the levels of Dlk1 and/or integrin beta-8 (Itgb8) in pericardial cultures, in vivo lesioned pericardium as well as in hearts after MI correlates with cardiac fibrosis or complications thereof it can be concluded that levels of DLK1 and/or ITGB8 in serum/plasma or pericardial fluid reflect the state of cardiac fibrosis or susceptibility here for. Thus, levels of Dlk1 and/or integrin beta-8 (Itgb8) in serum/plasma or pericardial fluid may likely be used to stratify cardiac patients in relation to risk, diagnosis, disease progression, and treatment.

Thus, in an embodiment, the sample is pericardial fluid. In another embodiment, the sample is a blood sample, such as serum and/or plasma.

### Alternative Aspects

An alternative aspect of the present invention relates to an inhibitor of Delta Like Non-Canonical Notch Ligand (Dlk1) or integrin beta-8 (Itgb8) for use in preventing and/or alleviating fibrotic scar tissue formation in the heart and/or around the heart, such as cardiac fibrosis in a subject, wherein said subject has suffered a heart condition.

In yet an aspect, the invention relates to a method for preventing and/or alleviating complications of a heart condition in a subject in need thereof, the method comprising administering to the subject an inhibitor of Delta Like Non-Canonical Notch Ligand (Dlk1) or integrin beta-8 (Itgb8), wherein said complications of the heart condition are fibrotic scar tissue formation in the heart and/or around the heart of a subject. In an embodiment, the inhibitor is administered by injection, such as intravenously. In another embodiment, the inhibitor is administered by intracardiac or intrapericardial fluid administration. In another embodiment, the inhibitor is administered within a week, such as within 72 hours of a myocardial infarction.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Test Subjects

### Human Samples

The following was collected for the Dlk1 analysis in the myocardium i) heart ventricle tissues from deceased patients (acute MI (n=6), chronic MI (n=6), hypertrophic (n=6), and normal (n=4) hearts), ii) aborted normal ventricle tissue from heart development (n=26; 91-280 days past fertilization (dpf)), and iii) heart biopsies of left ventricle (n=3) from patients undergoing valve surgery. For Dlk1 analysis of pericardial specimens plasma and pericardial fluids (n=127) as well as pericardial biopsies (n=12) from patients were obtained and elected for coronary artery bypass grafting (CABG) or cardiac valve replacement surgeries. Informed consent from all enrolled subjects were obtained when required. The protocols of the study were in accordance with the Declaration of Helsinki and were approved by the Medical Ethical Committee of the Region of Southern Denmark (Protocol: #S-20120065, #S-20180056, #S-20100044).

### Animals

When otherwise not indicated, C57BL/6 mice (Taconic, Ejby, DK) were used at the given developmental state. Plug breeding was checked in the morning and the evening, and 3-4 different litters at the given time points were used in all experiments. Dlk1^{-/-} mice shows complete absence of Dlk1 and is maintained by homozygous breeding. The αMHC-MerCreMer strain (A1cf^{(Myh6-cre)1Jmk/J}, The Jackson Laboratory, Bar Harbor, ME, USA) expresses inducible Cre in cardiomyocytes upon Tamoxifen treatment and is maintained by homozygous breedings. The WT1GFPCre (WT1^{tml(EGFP/cre) Wtp/J}, JAX stock #010911, The Jackson Laboratory, Bar Harbor, ME, USA) mouse expresses Cre in Wt1 (Wilms tumor 1 homolog) positive secondary heart field precursors, and is maintained by heterozygous breeding to C57bl/6 mice. The Dlk1^{fl/fl} (Dlk1^{flox/flox}) JI mouse was generated herein by a transgenic insert on the Rosa26-locus and consists of a CAG-promoter and full-length Dlk1 separated by a stop-sequence flanked by two loxP sites. Dlk1^{fl/fl}×WT1^{GFPCre} mice express Dlk1 in epicardial cells and descendants hereof such as fibroblasts and smooth muscle cells, while Dlk1^{fl/fl}×αMHC-MerCreMer express Dlk1 in cardiomyocytes in an inducible manner. Transgenics were backcrossed to C57BL/6 every third generation to avoid accumulation of non-specific traits. Tail or ear DNA was isolated using a DNeasy kit (Qiagen) and genotype analysis was performed by PCR amplification.

Tamoxifen (Sigma T5648, St. Louis, MO, USA) was dissolved in corn oil (Sigma C8267) at a concentration of 6 mg/mL and administered as intraperitoneal (i.p.) injections at a dose of 40 mg/kg per day for 5 consecutive days. To avoid tamoxifen mediated cardiac effects, tamoxifen was administered 4 weeks prior to experiment initiation, and confirmed at baseline that cardiac stress markers and heart functional were unaffected. Control animals received corn oil in equivalent quantities. All animal experiments were approved by, the Danish Council for Supervision with Experimental Animals (#2016-15-0201-00941).

### Cells

Mouse preadipocyte 3T3-L1 cells were obtained from ATCC and kept as recommended. Briefly, cells were plated at 600 cells/cm² 3 days before small interfering RNA (siRNA) transfection at day 3 with media replaced every 24 h (see Example 9). Cell culture medium consisted of Dulbecco's modified Eagle's medium (Lonza) supplemented with 10% calf serum (CS; Sigma-Aldrich) and 1% penicillinstreptomycin (PS; Lonza). Likewise, human epithelial cells from liver-HepG2 were plated at 85000 cells/cm₂ 24 hours before small interfering RNA (siRNA) transfection at day 1 and analysis at day 3 (see Example 9). Cell culture medium consisted of Dulbecco's modified Eagle's medium (Gibco) supplemented with 10% Fetal bovine serum (FBS; Sigma-Aldrich) and 1% penicillinstreptomycin (PS; Lonza). EPDCs were isolated from a given mouse litter by carefully dissecting the heart without disturbing the epicardium and pooled for each litter which then corresponded to one biological independent replicate. EPDCs were dissociated by gentle treatment with 0.3% Trypsin/DNase solution followed by centrifugation and red blood cell lysis. Harvested EPDCs were plated on ECM (E1270; Sigma-Aldrich) coated plates and cultured in DMEM (Dulbecco's Modified Eagle's Medium) supplemented with 20% FBS (Fetal Bovine Serum)/1% PS (Penicillin-Streptomycin) (all from Lonza). Purity was checked by flow cytometry. Total cell numbers were determined using a Beckman Coulter Counter Z2 fitted with a 100 μm aperture or a NucleoCounter® NC-200 (Chemometec, DK). EPDCs were used for experiments at passage 1-3. Briefly, EPDCs were plated 48 hours prior to transfection, and starved for 1 hour before 4 hours of transfection (20 nM siRNAs) thereafter medium was changed to DMEM/10% FCS±10 ng TGFβ (R&D Systems).

For isolation and culturing of fibroblasts, adult male C57BL/6J mice (Taconic, Denmark) were sacrificed by cervical dislocation. The hearts were carefully removed, cut into smaller cubes, and dissociated using the mouse and rat neonatal heart dissociation kit (Miltenyi Biotec; 4 samples, 2 hearts/sample) according to the manufacturer's instructions. Following dissociation, cells were washed in DMEM supplemented with 10% FBS and 1% PS and filtered through a 100 μm strainer. Viability and number of viable cells were determined using a Nucleocounter NC-200 (Chemometec, DK). The cardiac cells were cultured in DMEM supplemented with 10% FBS and 1% PS until they reached a confluency of 80% (96 h), passaged by trypsinization and frozen at P2 until initiation of the experiment. Cells were thawed and set up for experiment at P4 in 12-well plates. The day after seeding, cells were transfected in DMEM (no FBS and P/S) with a plasmid harboring full length Dlk1 (DLK1FL-PLHCX-HA), soluble Dlk1 (DLK1E-pLHCX-HA) or an empty control plasmid (pLHCX-HA; 1.25 μg/well, adjusted in molar concentration to DLK1FL-pLHCX-HA) using the Lipofectamine 2000 (Invitrogen) transfection reagent according to the manufacturer's recommendations. Four hours after transfection, the transfection reagents were removed and culture medium (DMEM supplemented with 10% FBS and 1% PS) added with—or without the addition of 10 ng/ml TGFβ (R&D Systems) or Dlk1 antibody (CC5+CC11; 5 μg/ml of each; generated in house) or its correlating isotype control (10 μg/ml). The experiment was terminated 48 h after transfection and processed for qRT-PCR.

### Example 2—Methods and Analysis

### Method for Introducing MI by Ligation of the Left Anterior Descending (LAD)

Mice were anaesthetized using 4% isoflurane (Baxter) and endotracheally intubated using the BioLite system (Braintree Scientific, Braintree, MA, US). Anaesthesia and respiration was maintained by assisted ventilation with 2.5% isoflurane and 100% oxygen using Minivent type 845 mouse ventilator (Hugo Sachs Elektronik Harvard Apparatus, March-Hugstetten, Germany). The heart was accessed by parting of the lower ribs and opening of the pericardial sac. Permanent ligation of the LAD was performed by placement of a single 8-0 prolene suture (Ethicon, Johnson&Johnson AB, Birkerød, DK) in the surrounding myocardium. Ligation of the LAD was confirmed visually by paling of the myocardium distal to the suture. The thoracic wall and skin were sutured, and the thoracic cavity was drained using a 23 G venflon. Body-temperature was kept between 36-37° C. avoiding hypothermia. Temgesic (RB Pharmaceuticals Limited, Slough, Berkshire, UK) was used for pain management. Mice that died immediately after surgery were not included in subsequent analyses. For heart localization in thorax, mice were killed by cervical dislocation, placed on the back, and all four legs fixed. The thorax was gently exposed without disturbing the heart, and the angle of the heart as compared to the head-tail orientation was determined manually using a simple angle goniometer. For heart to body ratio, the heart weight in mg of the body weight in g was calculated, though for neonatal mice, the heart to body ratio was determined by using an average of all hearts and mice in a given litter. Finally, for the heart shape, the overall shape and macroscopically features of the heart were assessed at dissection and evidenced by representative pictures.

### F-18-Fluorodeoxyglucose Positron Emission Tomography (FDG-PET)

To assess cardiac function, animals underwent FDG-PET imaging. All animals were anesthetized with a mixture of 1.5-2% isoflurane and 100% oxygen and injected via a tail vein catheter with a bolus of fluorodeoxyglucose (FDG) (30.5±3.5 MBq) and kept in anaesthesia for 15 minutes to prevent muscle uptake and hereafter returned to their cage. The animals were active 2 min after this mild anaesthesia. During the handling procedures and the complete FDG uptake period, animals were kept warm by a thermostat-controlled heating pad. Animals were re-anesthetized and placed in a supine position on a heated dedicated PET animal bed 25 min after injection of FDG. Electrocardiograph needle electrodes were placed left and right at the thorax and one at the left lower abdomen of the animals to collect the cardiac gating signal. During each imaging session the respiration, temperature and cardiac gating was monitored using the BioVet system (M2M Imaging, Cleveland, Ohio, US). FDG-PET was performed using a small animal PET scanner (INVEON, Siemens pre-clinical solutions, Knoxville, TN, US) covering the entire mouse with an axial view field of 127 mm. Static PET acquisition was performed for 30 min after an FDG uptake period of 31±4 min. The list-mode PET data were framed into 12 cardiac gates and reconstructed using an OSEM3D/MAP algorithm (4 OSEM3D and 18 MAP iterations, requested resolution 0.1 mm) using the Siemens INVEON pre-clinical software. FDG-PET images were analysed by a blinded medical physicist with high experience in using the software (Cedars-Sinai Medical Center, Los Angeles, CA, USA). This software allows for automatic processing with only alignment of the heart axes as a variable thus minimizing the bias of the analysis. Accordingly, functional parameters (EF (%), EDV (μL), ESV (μL)) were established, and stroke volume (EDV-ESV) was calculated. Moreover, wall motion, stress perfusion and thickness were reported by the 20-segmentation model.

### Histology and Immunohistochemistry

Dissected hearts were embedded in Tissue-Tek (Sakura Finetek Europe, Alpena aan den Rijn, NL) and snap-frozen using isopentane (Sigma-Aldrich). For heart development series, hearts were sectioned frontally, and sliced throughout in steps to reveal all parts of the hearts (three different hearts derived from individual litters were examined). For scar-size quantification, hearts were cross-sectioned in steps (50 sections each) starting from the apex towards the base until the scar tissue was absent as visualized by haematoxylin-eosin (HE) and Masson Trichrome (MT) staining. The amount of scar tissue and viable myocardium was quantified at each step using Adobe Photoshop, where the observer was blinded to the group and genotype. For HE staining, sections were fixed in 4% neutral buffered formalin (NBF) for 5 min before staining with Mayers hematoxylin with citric acid (Amplicon, Odense, DK) and eosin 0.2% (Sakura). Sections for MT staining, were fixed for 1 hour in 4% NBF and subsequently prepared in Bouins solution (Sigma-Aldrich) overnight. Staining was performed using Weigerts Iron Hematoxylin and Trichrome stain (Masson) kit (Sigma-Aldrich). For IHC, sections were fixed in 4% NBF (10 min) and blocked using 2% bovine serum albumin (BSA) in TBS. Primary antibodies were diluted in 1% BSA. Primary antibodies used included rabbit anti-mouse DLK17 (1:2000, in-house), rabbit anti-human DLK139 (1:500, in-house), mouse anti-mouse DLK140 (C5/C11; μg/mL, in-house), mouse anti-MYH1 (Sarcomeric myosin, 1:200, DSHB), rabbit anti-Islet 1 (1:750, Abcam), mouse anti-Islet 1 (1:25-50, DSHB), goat anti-Desmin (1:50, Santa Cruz Biotechnology, Dallas, TX, USA), mouse-anti alpha smooth muscle actin (aSMA, 1:2-400; Sigma-Aldrich), rabbit anti-Wilms tumor 1 (WT1, Abcam, 1:50), rat anti-Flk1 (1:50, eBiosciences), rat anti-Pecam1 (CD31, 1:50, BD Pharmingen), rat anti-CD34 (1:50, BD Pharmingen), rat anti-E-cadherin (Cdh5, 1:700, BD Biosciences), rat anti mouse PDGFRa (1:50, Abcam), goat anti-DDR2 (1:40, Santa Cruz Biotechnology), mouse anti-Vimentin (Vim, 1:40, Sigma-Aldrich), rat anti-Laminin (1:50, Sigma-Aldrich), rat-anti-mouse CD45 (1:50, BD Pharmingen), mouse-anti-TroponinT (1:2-400, Sigma-Aldrich), Cytokeratin 19 (1:50, CK19, DSHB), goat anti-rat CD31 (1:50, Santa Cruz Biotechnology). Secondary antibodies (all 1:200, Molecular Probes, Eugene, OR, USA) used were either conjugated with Alexa 488-, Alexa-555 or Alexa 647. Mounting medium contained DAPI (Vectashield, Vector Labs, Burlingname, CA, USA). For Wheat germ agglutinin (WGA) stainings cryosections were fixed and blocked as described for IHC and incubated for 90 min with WGA Alexa fluor 488 conjugate (Molecular Probes) diluted in TBS to 10 μg/mL. For paraffin embedded sections, stainings were performed by use of Envision+ (DAKO) and carbazole for visualization. Microscopic examinations of fluorescent stained sections were performed using a Leica DMI4000B Cool Fluo Package instrument equipped with a Leica DFC340 FX Digital Cam and a Leica DFC 300 FX Digital cam (Leica Microsystems, Ballerup, DK). In all experiments, camera settings and picture processing were applied equally to samples and controls. Histological sections were examined using a Leica M80 stereomicroscope with Leica IC80 HD digital cam (Leica Microsystems). Photoshop (versions up to 21.2.2, Adobe systems Inc.) was used for picture processing.

Relative Quantitative Real Time PCR (qRT-PCR)

Total RNA was extracted from cells or tissues by Trizol (Thermo Fisher Scientific) as in general and for cDNA synthesis 0.3-0.4 μg of total RNA was reverse transcribed with High Capacity cDNA RT kit (Thermo Fisher Scientific). Quantitative real-time polymerase chain reactions (qRT-PCR) using customized primer sets were performed and run on a 7900HT Fast Real-time PCR system (Applied Biosystems) or a QUANTSTUDIO 7 FLEX instrument (Thermo Fisher Scientific). As recommended by others, robust and valid qRT-PCR data was obtained by normalizing the raw data against multiple stably expressed endogenous control genes as determined by the qBase Plus platform.

### Flow Cytometry to Determine Dlk1

Cells were detached, washed twice in HBSS/10% CS/1% PS, and fixed for 30 min on ice in 1% normal buffered formaldehyde. Fixed cells were washed three times and stored at 4° C. in HBSS/5% CS/1% PS/0.05% NaN₃ until analysis. Cells were immunostained with rabbit a-mouse Dlk1 antibody (0.45 mg/mL) generated in house or with rabbit immunoglobulin (Ig) G (control; Santa Cruz Biotechnology) for 30 min on ice. After washing twice, samples were incubated with Alexa 488-conjugated donkey a-rabbit IgG (1:200; Molecular Probes, Invitrogen) for 30 min on ice followed by two washes before flow cytometric analysis using a BD FACSCalibur Instrument. The BD FACSDiva software (version 5.0.1; BD Biosciences, San Jose, CA) was used to analyse flow cytometric results. Debris was excluded from the analysis by gating in the forward and side scatter as previously described. The relative Dlk1M levels were calculated as fold geometric Alexa-488 fluorescence (sample/control) of all live cells on a given day. In parallel, the percentage of Dlk1-positive cells in this live cell gate was measured by gating using the control.

### ELISA

For mice, blood was collected in T-MLH (heparinized) tubes (CAPIJECT capillary micro collection tubes, Tokyo, Japan), and plasma was obtained for further analysis. Corresponding human blood and pericardial fluid (PF) samples were collected. Quantification of human and mouse DLK1 was performed using developed sandwich ELISAs. Briefly, for the human DLK1 ELISA a polyclonal monospecific rabbit anti-human DLK1 antibody was used as catcher antibody, while a biotinylated F (ab) 2 fragment of the same antibody served as detector antibody. The mouse DLK1 ELISA was based on two monoclonal antibodies, CC5 and CC11 as catcher/detector antibodies respectively. Both assays utilized amniotic fluid (human or mouse as appropriate) as a source of native DLK1 for calibrators and quality controls. Horseradish peroxidase-labelled streptavidin (Invitrogen, Camarillo, CA, USA) was used as conjugate and Ortho-Phenylenediamine (Kem-En-Tec Diagnostics a/s, Taastrup, Denmark) as substrate. Absorbance was measured at 490 nm.

### Statistical Analysis

All data are presented in the figures, and each analysis consisted of at least three independent experiments designated n, and when indicated n* refers to number of animals in each experiment. Statistical significance of the difference between means was determined by either two-tailed t-tests, or by one- or two-way ANOVA followed by appropriate post-hoc tests as indicated. The choice of test included normality of data. Clinical values were presented using the median and interquartile range (IQR). Associations between DLK1 and clinical parameters of interest were evaluated by correlation analysis using Spearman's rank correlation coefficient. The GraphPad Prism (9.0 Mac version) software was used for all statistical calculations. *P<0.05, **P<0.01, ***P<0.001, ****P<0.0001, ns (not significant).

### Example 3—Dlk1 is Secreted from the Peri-/Epicardium into the Pericardial Fluid of Human Subjects

Normal human embryonic and adult hearts were analysed, and it was found that Dlk1 mRNA levels increase during human heart development and then declines to be become nearly abolished in normal adult ventricles (FIG. 1A). This was confirmed by immunohistochemistry showing that Dlk1 protein localizes to many non-cardiomyocytes in the developing human heart, whereas Dlk1 is rarely detected in the adult ventricle of normal individuals, although rare non-myocyte localized Dlk1+ cells was observed in association with vessels mainly. Moreover, Dlk1+ cells were more or less absent in the myocardium from patients suffering from acute MI (n=7), chronic MI (n=6), or cardiac hypertrophy (n=8). On the contrary, in pericardial specimens from another cohort of patients undergoing cardio-thoracic surgery for residual cardiovascular disease, a substantial and consistent Dlk1 expression (n=12) in the pericardium was found (FIG. 1B). Dlk1 localized to discrete stretches of mesothelial cells and many vascular structures within this compartment as well as in some stromal cells interspersed within the collagen matrix (FIG. 1B). Pericardial fluid (PF) and venous plasma was then collected from a cohort of 127 patients undergoing valve or CABG surgery to test if soluble Dlk1 (sDlk1) indeed was secreted into the PF. No differences in sDlk1 levels were observed between sexes for neither plasma nor PF, and the levels did not correlate to “continuous” classical risk factors, possibly due to the extensive treatment of the patients. The median sDlk1 in venous plasma (26.10 μg/L (11.1 (IQR))) of these cardiovascular patients was within the normal range, whereas sDlk1 in the PF (median 45.7 (34.7 (IQR)) μg/L) compartment was significantly (P<0.0001) higher (FIG. 2A). The mean sDlk1 ratio ([PF]/[P]) was 2.041±1.3 (SD) fold higher in PF than in venous plasma and the sDlk1 in the two compartments showed a strong linear correlation (FIG. 2B).

Conclusion: These data thus suggest that in humans, Dlk1 is expressed in the peri-/epicardial cell lineage during development, but in adulthood becomes restricted to the pericardial compartment where it is secreted into the PF.

### Example 4—Dlk1 is Expressed in the EPDC Lineage During Mouse Heart Development

The temporal and spatial expression of Dlk1 in the heart of C57bl/6 mice from E9.0 until adulthood was explored. Full length Dlk1 as well as Dlk1^{Soluble} mRNA (Dlk1 variants that comprise the protease site) expression increased during mouse heart embryogenesis, but ceased in neonates, and were almost completely abolished in adult mouse hearts (FIG. 3A and FIG. 3B) in agreement with the human Dlk1 expression profile (FIG. 1A). At E9.0-9.5, Dlk1 protein localized to the pariethal pericardium, the proepicardial organ, some interstial non-myocytes (myosin negative) in the right ventricle and numerous cells in the outflow tract (OFT). At E10.5-E11, this Dlk1 expression pattern was extended to comprise also the visceral pericardium, the left ventricle, and a few cells lining myocytes throughout the atria. At E10.5, the OFT residing Dlk1+ cells co-expressed the EPDC markers Islet 1 and WT1 (Wilms tumor protein), but also structural myogenic proteins like Myosin, alpha smooth muscle actin (aSMA), and Desmin known to be expressed in this transient structure that forms the base of the aorta. Within the ventricle wall, Dlk1 was expressed in relation to the developing CD31+/CD34+/FLK1+ capillary vasculature, but did not co-localize with cardiomyocyte proteins. A careful examination of the peri-/epicardium revealed that Dlk1+ cells co-express aSMALow, PDGFRa, E-Cadherin, and WT1, but not Islet 1. The epicardium and pericardium is often considered as united in the mouse, but based on E-Cadherin being in the pericardium, but not the epicardium, it seems that Dlk1 localized to both layers. From E12.5 until postnatal day three (P3), Dlk1 expression was high in the mesenchyme surrounding the larger vessels, but it was also markedly expressed in the pariethal-/visceral pericardium, small interstitial cells in the ventricles and small cells lining the myocytes in the atria. The vascular associated Dlk1 expression was substantial at E16.5, where Dlk1 seemed to mark a large fraction of the developing CD31+/CD34+/FLK1+ capillary vasculature within the myocardium, but not in large vessels where it resided in the fibroblasts composed adventitia. However, this was not the case at postnatal day 3 (P3), where Dlk1 besides the expression in the peri-/epicardium, merely was expressed in interstitial long-strected cells with a fibroblast like CD34+/DDR2+/Laminin+/Vimentin+ phenotype. At P14, only a few Dlk1+ cells were scattered around in the ventricles and atria as well as in mesenchymal structures such as papilliary muscles, AV-junction, AV/aortic valves, and septum. Large vessels were Dlk1 negative, but surrounding adventitia Dlk1 positive. The epicardium and to a lesser extent the pericardium remained Dlk1 positive at this stage, but at P98, referring to adulthood, Dlk1 were more or less absent throughout the heart, except for a very few non-myocytic cells and some long-stretched Dlk1+ cells lining the pericardium, the interior of the aorta, and the surrounding mesenchyme.

Conclusion: The mouse data supports the human Dlk1 expression pattern described above and suggest that a first wave of Dlk1+ cells from E10.5 to E16.5 marks early multipotent progenitors of the EPDC cell lineage including vascular cells, whereas a second neonatal wave (around P3) of Dlk1+ cells mainly comprises cardiac fibroblasts descendants. However, in adulthood Dlk1 is mainly restricted to the peri-/epicardial cells.

### Example 5—Dlk1 Promotes Fibroblast Differentiation and Expansion Through Itgb8

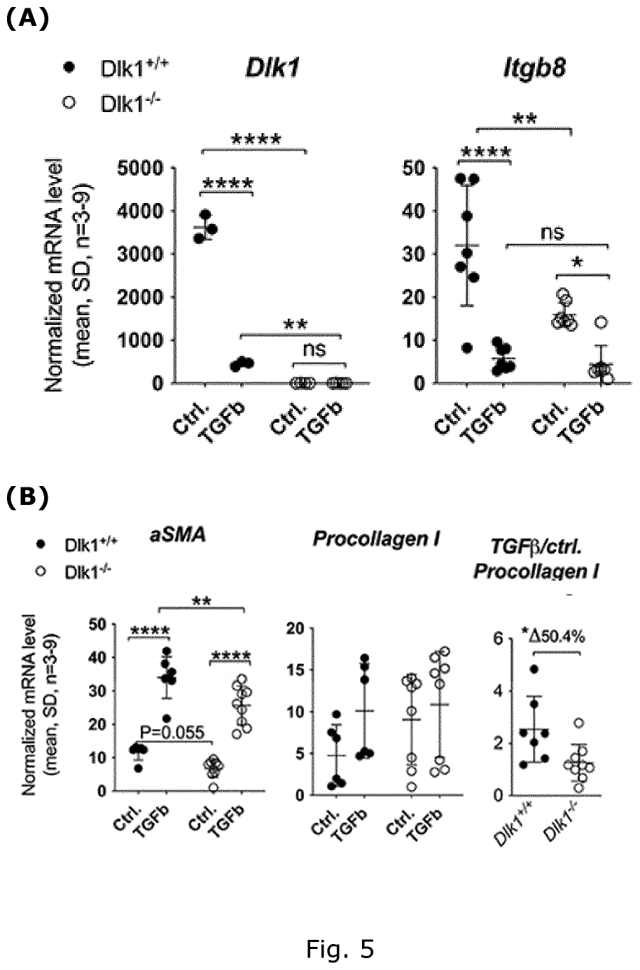

The number of fibroblasts markedly expands in the neonatal period. Therefore, mouse peri-/epicardium were stripped and pure in vitro cultures of Dlk1^{+/+} and Dlk1^{-/-} neonatal EPDCs were established to further explore the role of Dlk1 in EPDC EMT. The expression of Dlk1 in Dlk1^{+/+} EPDCs seemed dynamic since, Dlk1 negative sorted Dlk1^{+/+} EPDCs gave rise to Dlk1 positive EPDCs, which may explain why only 83.7±9.9% (n=4) of Dlk1^{+/+} EPDCs were positive for Dlk1 at a given time point. Moreover, a slightly increased proliferation potential of Dlk1 deficient EPDCs was found. Global gene expression profiling of Dlk1^{+/+} and Dlk1^{-/-} EPDCs, using high stringency (SAM and LIMMA test) revealed a small number of genes robustly affected by Dlk1. Of these, substantially decreased levels in Dlk1^{-/-} EPDCs of the Integrin β8 subunit (ITGB8) were found. ITGB8 is a recently demonstrated activator of transforming growth factor β (TGFβ). Overall Itgb8 levels were similar in a large range of adult Dlk1^{+/+} and Dlk1^{-/-} tissues excluding that the observed Itgb8 downregulation in Dlk1^{-/-} EPDCs was due to an artefact on chromosome 12 where both genes are located. Moreover, Itgb8 was reduced in Dlk1^{-/-} hearts during cardiac development, with a substantial reduction at E16.5 (FIG. 4) where Dlk1 levels peaks in Dlk1^{+/+} hearts (FIG. 3A and FIG. 3B). Next, neonatal Dlk1^{+/+} and Dlk1^{-/-} EPDCs were stimulated with TGFβ, a major EMT inducer, and it was found that fibroblasts differentiation (increase in aSMA and Procollagen I) was inhibited in Dlk1^{-/-} EPDCs as compared to Dlk1^{+/+} EPDCs (FIG. 5A and FIG. 5B). To assess the specific contribution of Itgb8 in this EMT process, Itgb8 in Dlk1^{+/+} EPDCs were effectively knocked down and a 30- and 37% reduction in Procollagen I was observed, but no effect on Dlk1 itself (FIG. 6). This indicates that Itgb8 mediates EMT of EPDCs, and that Dlk1 is upstream of Itgb8. The sequences used for knockdown of Itgb8 were:

| SEQ ID NO: | Sequence: | Strand: |
|---|---|---|
| 23 | 5′CAAGCUAUCUGCGAAUAUUTT 3′ | + |
| 24 | 5′AAUAUUCGCAGAUAGCUUGGG 3′ | − |

Conclusion: Together these data support that Dlk1 increases the epithelial to mesenchymal transition (EMT) of EPDCs and suggest that this process involves regulation of Itgb8 affecting TGFβ function.

### Example 6—Dlk1 Aggravates Scar Size after Myocardial Infarction

Since EPDC derived fibroblast expansion and differentiation are major events in myocardial remodelling and scarring after MI, the next aim was to elucidate whether Dlk1 plays a role therein. MI was introduced by ligation of the left anterior descending (LAD) coronary artery of the mice. Full length Dlk1 and soluble Dlk isoforms increased in both LAD and sham hearts (placebo surgery) at day 7 (FIG. 7A and FIG. 7B). Dlk1 protein in the circulation was assayed following MI and it was found that circulating Dlk1 protein, as in agreement with Dlk1 mRNA levels, increased at day 7 in both Sham and LAD mice (FIG. 7C).

Then, LAD surgery was performed in three sets of Dlk1 transgenic mice and their corresponding controls to test whether Dlk1 impact cardiac remodelling following MI:
i) Dlk1^{-/-} (Dlk1 knockout),
   Dlk1^{+/+} as control.
ii) Dlk1^{fl/fl}×WT1^{GFPCre} (EPDC lineage Dlk1 overexpression),
   Dlk1^{fl/fl}×C57bl/6 as control, and
iii) Dlk1^{fl/fl}×αMHC^{Cre/+ Tam} (Cardiomyocyte Dlk1 overexpression)
   Dlk1^{fl/fl}×αMHC^{Cre/- Tam} as the control.

Myocardial function was assessed by Positron-emission tomography (18FDG-PET). At 8 weeks after MI, histological analysis of serial heart sections showed that scar size was decreased with 33.4% in Dlk1^{-/-} hearts (FIG. 8A), but increased in Dlk1^{fl/fl}×αMHC^{Cre/+ Tam} with 21.6% (FIG. 8B) and tended (p=0.07) to be increased with 24.8% in Dlk1^{fl/fl}×WT1^{GFPCre} hearts (FIG. 9).

Conclusion: As for the EMT promoting role during heart development, these data showed that the presence of dlk1 also after MI in adulthood negatively affects cardiac remodelling by enhancing fibrosis. This is particular important since high amounts of Dlk1 in the human pericardium and the pericardial fluid was observed (FIG. 1A) and which potentially enhance fibrosis and collagen accumulation after MI.

### Example 7-Pericardial Sac Lesions Increases Dlk1 and Itgb8 Levels that Activates TGFβ Whereby Collagen Production and Heart Fibrosis is Increased

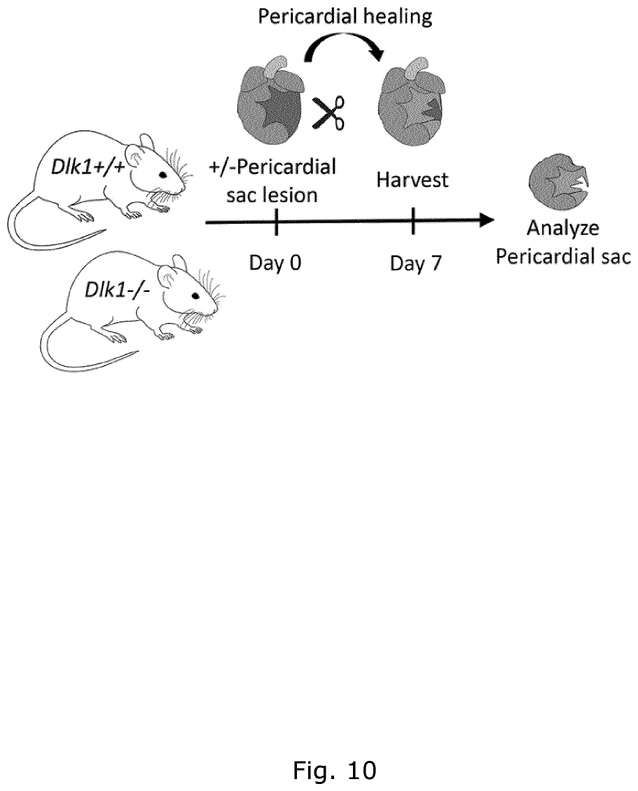

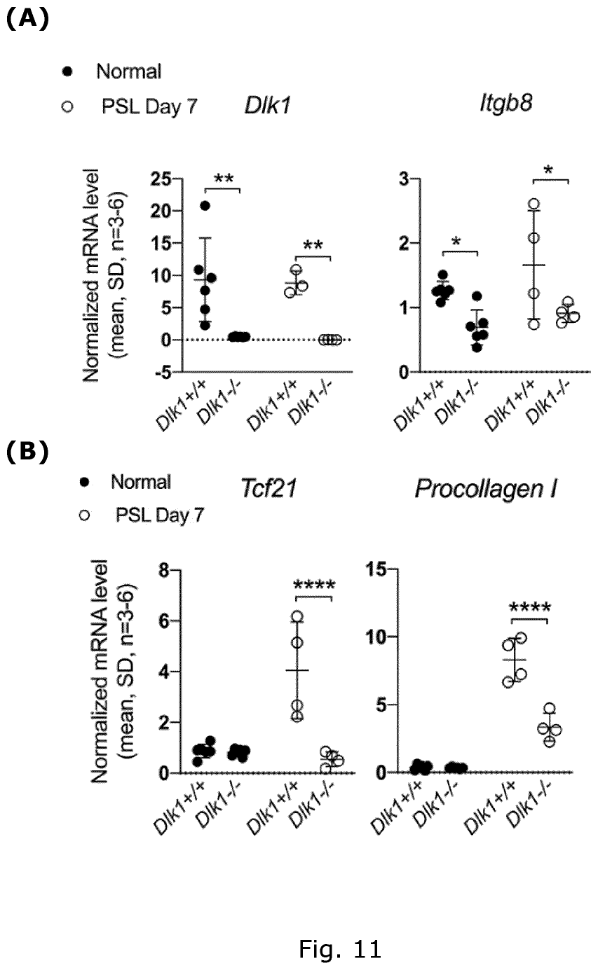

It was tested whether reactivation of the pericardium alone triggers a Dlk1 dependent EMT response in the pericardium. Pericardial sac lesions in Dlk1^{+/+} and Dlk1^{-/-} animals were performed and the dissected pericardium analysed (FIG. 10). Dlk1 was as expected present exclusively in Dlk1^{+/+} pericardial specimens but did not differ between injured and uninjured pericardium (FIG. 11A). In agreement, Itgb8 was higher in Dlk1^{+/+} versus Dlk1^{-/-} pericardium but the level was also independent of injury as with Dlk1.

In contrast, the amount of Procollagen I and the epicardial fibroblast progenitor transcription factor (Tcf21) were decreased in Dlk1^{-/-} as compared to Dlk1^{+/+} specifically in the injured pericardium (FIG. 11B). It shows that Dlk1 affects heart fibrosis through increased procollagen I expression in the pericardium and in interstitial cardiac fibroblasts.

Conclusion: These results point to a mechanism where Dlk1 sustain Itgb8 levels as part of normal homeostasis. However, in times of remodelling, when TGFβ is present, this mechanism amplify collagen production and fibrosis.

### Example 8-Inhibition of Dlk1^{+/+} in Fibroblast Cells Decreases the TGFβ Induced Procollagen I Expression

The in vivo scenario of interstitial cardiac fibroblast (iCF) myofibroblast conversion after MI was mimicked and Dlk1's effect tested. Soluble Dlk1 was as expected absent in primary derived adult iCFs, but could be successfully expressed transiently independent of TGFβ. iCFs overexpressing Dlk1 increased Procollagen I expression upon TGFβ stimulation as expected. However, this response was specifically inhibited by adding an anti-Dlk1 antibody (mouse monoclonal anti-mouse Dlk1, clone CC-5), whereas an isotype matched control did not affect iCF myofibroblast conversion (FIG. 12).

Conclusion: These data exemplify that pericardial Dlk1 works to enhance scarring after MI. Targeting Dlk1 (in this example with an antibody binding to Dlk1) in the pericardium or the pericardial fluid may therefore offer a new venue for reducing cardiac fibrosis after MI.

### Example 9—Knockdown of Dlk1 Using siRNA

Two specific siRNAs were designed to differentially target either all Dlk1 mRNA splice variants (SEQ ID NO: 25; SEQ ID NO: 26) or only Dlk1 mRNA splice variants containing coding sequences for the protease site for extracellular cleavage (SEQ ID NO: 27; SEQ ID NO: 28).

| SEQ ID NO: | Sequence: | Strand: |
|---|---|---|
| 25 | 5′ AGAUCGUAGCCGCAACCAATT 3′ | + |
| 26 | 5′ UUGGUUGCGGCUACGAUCUCA 3′ | − |
| 27 | 5′ UCCUGAAGGUGUCCAUGAATT 3′ | + |
| 28 | 5′ UUCAUGGACACCUUCAGGATG 3′ | − |

Silencer Select Negative control (no. 4390846; Ambion) was used as control, and transfections were performed for 4 h using Lipofectamine 2000 (Invitrogen). Robust and reproducible Dlk1 knockdown siRNA and conditions were established.

Knockdown of Dlk1 mRNA in preadipocyte cells (3T3-L1 cells (mouse)) by said specific siRNA was measured by qRT-PCR as shown in FIG. 13A and FIG. 13C, whereas knockdown of the membrane bound/tethered Dlk1 (Dlk1^{M}) was measured by Flow cytometry as depicted in FIG. 13B. Soluble Dlk1 knockdown was detected by ELISA of the medium (FIG. 13D).

FIG. 13E shows use of the same siRNAs for efficient knockdown of Dlk1 in mouse peri-epicardial cells in a culture as determined by qRT-PCR.

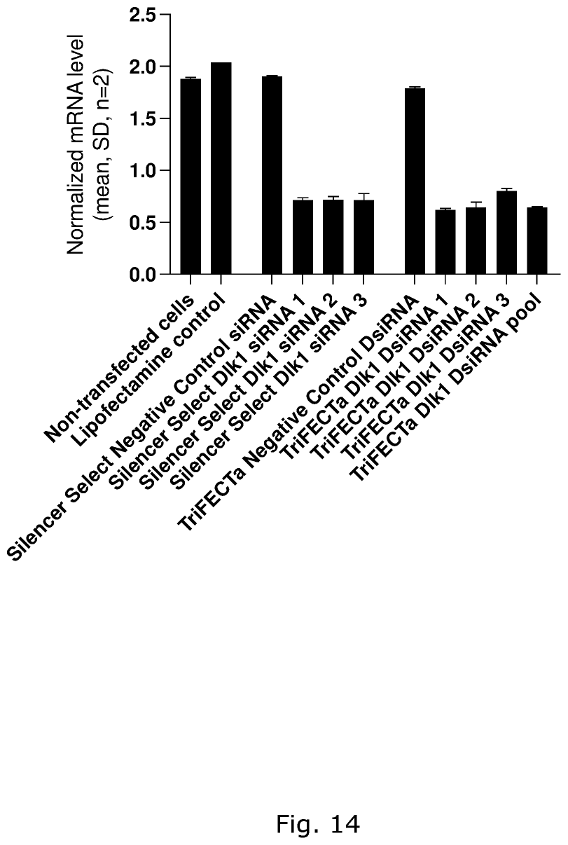

FIG. 14 shows use of siRNAs designed for efficient knockdown of Dlk1 in human HepG2 cells, which are similar to mesothelial cells in the pericardium as both are epithelial cells. The knockdown was validated in a culture and evaluated by qRT-PCR and corresponding controls (see also Example 1).

The designed siRNAs were:
a silencer select Dlk1 siRNA 1 (pair of SEQ ID NO: 11 and 12)
a silencer select Dlk1 siRNA 1 (pair of SEQ ID NO: 13 and 14)
a silencer select Dlk1 siRNA 1 (pair of SEQ ID NO: 15 and 16)
a TriFECTa Dlk1 DsiRNA 1 (pair of SEQ ID NO: 17 and 18)
a TriFECTa Dlk1 DsiRNA 2 (pair of SEQ ID NO: 19 and 20), and
a TriFECTa Dlk1 DsiRNA 3 (pair of SEQ ID NO: 21 and 22).

Conclusion: The designed siRNA are effective at knocking down Dlk1 mRNA of all forms (soluble, tethered etc.) in both mouse preadipocyte and mouse peri-epicardial cells as well as in human cells. Effects mediated by Dlk1 protein may therefore be lowered/inhibited by use of said siRNA.

### Example 10—In Vivo Delivery of siRNAs in the Pericardial Compartment

SIRNAs were designed to target Dlk1 or Itgb8 and preliminary tested for their ability to be applied in the pericardial compartment of wildtype mice with subsequent analysis of Dlk1 or Itgb8 knockdown.

The designed siRNAs were:
An in vivo grade silencer select Dlk1 siRNA (pair of SEQ ID NO: 25 and 26)
An in vivo grade silencer select Itgb8 siRNA (pair of SEQ ID NO: 23 and 24)
Silencer Select Negative control (no. 4390846; Ambion)

Upon open thoracic surgery wildtype mice were injected with siRNA diluted in invivofectamine 3.0 reagent into the pericardial compartment. Blood samples were collected before surgery and at day 7 for DLK1 protein measurements by ELISA, whereafter the pericardium was dissected at day 7 for analysis of Dlk1 and Itgb8 mRNA levels by qRT-PCR.

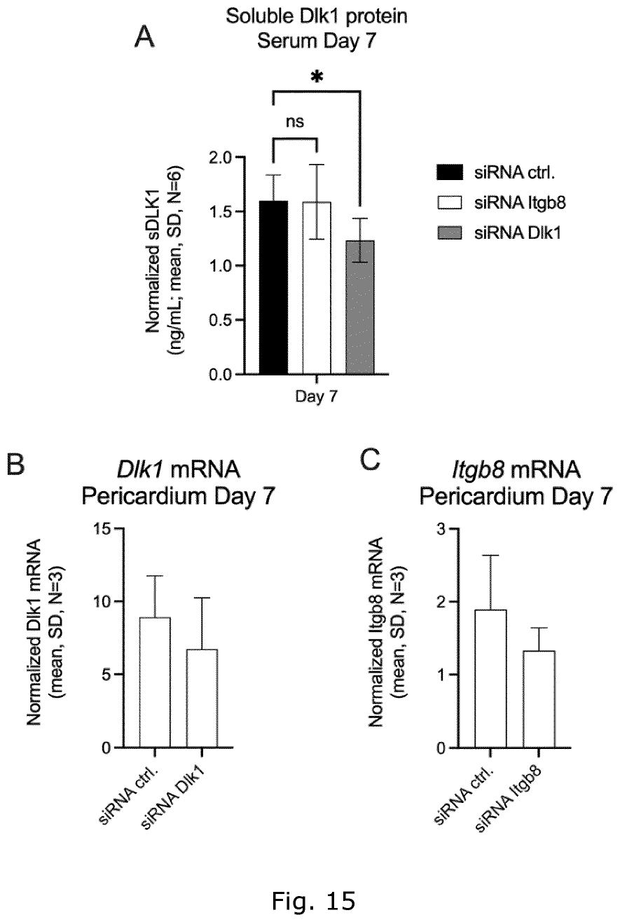

The preliminary data suggest that Dlk1 and Itgb8 targeting siRNAs (without induction of a lesion) can be applied directly to the pericardium with a lowering of Dlk1 of ITGB8 levels (FIG. 15).

Conclusion: The designed siRNA may be applied in the pericardium with knocking down Dlk1 and Itgb8 mRNAs and proteins in the mouse. Effects from Dlk1 and Itgb8 may thus be lowered by delivery of siRNAs to the pericardial space.

1. A method for preventing and/or alleviating complications of a heart condition in a subject in need thereof, the method comprising administering to the subject an inhibitor of Delta Like Non-Canonical Notch Ligand 1 (Dlk1) or integrin beta-8 (Itgb8);
wherein said complications of the heart condition are fibrotic scar tissue formation in the heart and/or around the heart of a subject;
wherein the inhibitor is selected from the group consisting of
an oligonucleotide targeting the DLK1 gene, the Itgb8 gene or transcripts thereof; and
an anti-DLK1 antibody or an anti-Itgb8 antibody.

2. The method according to claim 1, wherein the heart condition is an acute condition or a chronic condition.

3. The method according to claim 1, wherein the heart condition is selected from the group consisting of myocardial infarction, aortastenose, heart surgery, age-related heart disorders, and cancer of the heart.

4. The method according to claim 1, wherein the heart condition is myocardial infarction.

5. The method according to claim 1, wherein the heart condition is heart surgery.

6. The method according to claim 1, wherein the heart condition is an acute heart condition.

7. The method according to claim 1, wherein the inhibitor is delivered to the pericardium and/or pericardial fluid of the heart.

8. The method according to claim 1, wherein the subject is a mammal.

9. The method according to claim 1, wherein the oligonucleotide is selected from the group consisting of an antisense RNA (asRNA), antisense DNA (asDNA), guide RNA (gRNA), and small interfering RNA (siRNA).

10. The method according to claim 1, wherein the oligonucleotide is complementary to the DLK1 gene, the Itgb8 gene or transcripts thereof.

11. The method according to claim 1, wherein the antibody is selected from the group consisting of a polyclonal antibody, a monoclonal antibody, an antibody, wherein the heavy chain and the light chain are connected by a flexible linker, an Fv molecule, an antigen binding fragment, a Fab fragment, a Fab′ fragment, a F(ab′)2 molecule, a fully human antibody, a humanized antibody, a nanobody and a chimeric antibody.

12. The method according to claim 1, wherein the antibody is a neutralizing antibody.

13. The method according to claim 1 wherein the inhibitor is administered by intracardiac or intrapericardial fluid administration.

14. The method according to claim 1, wherein the inhibitor is administered within a week of a myocardial infarction.

15. The method according to claim 1, wherein the inhibitor is administered within a week from the beginning of a heart surgery.

16. The method according to claim 1, wherein the method comprises administering a pharmaceutical composition to the subject, wherein the composition comprises one or more inhibitors according to any of the preceding claims and one or more pharmaceutical acceptable excipients and/or carriers.

17. A method for determining a likely effect of a treatment to prevent and/or alleviate cardiac fibrosis, the method comprising:
determining the level of Dlk1 and/or integrin beta-8 (Itgb8) protein in a sample of pericardial fluid or blood, such as serum and/or plasma, which has been obtained from a subject who suffers from a heart condition;
comparing said determined Dlk1 and/or Itgb8 level to a corresponding reference level; and
determining that said subject will
likely not benefit from treatment to prevent or alleviate cardiac fibrosis or complications thereof, such as an inhibitor of Dlk1 or Itgb8, if said level is lower than said reference level, or
likely benefit from treatment to prevent or alleviate cardiac fibrosis or complications thereof, such as an inhibitor of Dlk1 or Itgb8, if said level is equal to or higher than said reference level.

18. A method for determining if a subject is at risk of suffering from cardiac fibrosis, the method comprising determining the level of Dlk1 and/or integrin beta-8 (Itgb8) protein in a sample of pericardial fluid or blood, such as serum and/or plasma, which has been obtained from a subject;
comparing said determined Dlk1 and/or Itgb8 level to a corresponding reference level; and
determining that said subject
likely suffers from cardiac fibrosis or complications thereof, if said level is equal to or higher than said reference level; or
likely not suffering from cardiac fibrosis or complications thereof, if said level is lower than said reference level.

19. The method according to claim 17, wherein the sample is pericardial fluid.

20. The method according to claim 17, wherein the sample is a blood sample, such as serum and/or plasma.


SEQUENCE LISTING (28 sequences)

SEQ ID NO: 1 - DNA, 25 bp, Artificial Sequence
  antisense oligonucleotide
  RNA (1)..(23)
  DNA (24)..(25)
ccugcaacccccaaaauggauuctg

SEQ ID NO: 2 - DNA, 27 bp, Artificial Sequence
  Antisense oligonucleotide
  RNA (1)..(27)
cagaauccauuuuggggguugcaggcc

SEQ ID NO: 3 - DNA, 25 bp, Artificial Sequence
  Antisense oligonucleotide
  RNA (1)..(23)
  DNA (24)..(25)
cucucuuaaugcaugauacagaata

SEQ ID NO: 4 - DNA, 27 bp, Artificial Sequence
  Antisense oligonucleotide
  RNA (1)..(27)
uauucuguaucaugcauuaagagagag

SEQ ID NO: 5 - DNA, 25 bp, Artificial Sequence
  Antisense oligonucleotide
  RNA (1)..(23)
  DNA (24)..(25)
cuugugucaaaucuggugaacgcta

SEQ ID NO: 6 - DNA, 27 bp, Artificial Sequence
  Antisense oligonucleotide
  RNA (1)..(27)
uagcguucaccagauuugacacaagag

SEQ ID NO: 7 - DNA, 25 bp, Artificial Sequence
  Antisense oligonucleotide
  RNA (1)..(23)
  DNA (24)..(25)
cucucucuuaaugcaugauacagaa

SEQ ID NO: 8 - DNA, 27 bp, Artificial Sequence
  Antisense oligonucleotide
  RNA (1)..(27)
uucuguaucaugcauuaagagagagaa

SEQ ID NO: 9 - DNA, 25 bp, Artificial Sequence
  Antisense oligonucleotide
  RNA (1)..(23)
  DNA (24)..(25)
aaauaaguauguuauucuaaaauct

SEQ ID NO: 10 - DNA, 27 bp, Artificial Sequence
  Antisense oligonucleotide
  RNA (1)..(27)
agauuuuagaauaacauacuuauuucu

SEQ ID NO: 11 - DNA, 25 bp, Artificial Sequence
  Antisense oligonucleotide
  RNA (1)..(23)
  DNA (24)..(25)
cucuuaaugcaugauacagaauaat

SEQ ID NO: 12 - DNA, 27 bp, Artificial Sequence
  Antisense oligonucleotide
  RNA (1)..(27)
auuauucuguaucaugcauuaagagag

SEQ ID NO: 13 - DNA, 25 bp, Artificial Sequence
  Antisense oligonucleotide
  RNA (1)..(23)
  DNA (24)..(25)
aaaauggauucugcgaggaugacaa

SEQ ID NO: 14 - DNA, 27 bp, Artificial Sequence
  Antisense oligonucleotide
  RNA (1)..(27)
uugucauccucgcagaauccauuuugg

SEQ ID NO: 15 - DNA, 25 bp, Artificial Sequence
  Antisense oligonucleotide
  RNA (1)..(23)
  DNA (24)..(25)
ggcuucucaggcaauuucugcgaga

SEQ ID NO: 16 - DNA, 27 bp, Artificial Sequence
  Antisense oligonucleotide
  RNA (1)..(27)
ucucgcagaaauugccugagaagccag

SEQ ID NO: 17 - DNA, 21 bp, Artificial Sequence
  Antisense oligonucleotide
  RNA (1)..(19)
  DNA (20)..(21)
cuuaaugcaugauacagaatt

SEQ ID NO: 18 - DNA, 21 bp, Artificial Sequence
  Antisense oligonucleotide
  RNA (1)..(19)
  DNA (20)..(21)
uucuguaucaugcauuaagag

SEQ ID NO: 19 - DNA, 21 bp, Artificial Sequence
  Antisense oligonucleotide
  RNA (1)..(19)
  DNA (20)..(21)
caaacgcaaugcaaaaacatt

SEQ ID NO: 20 - DNA, 21 bp, Artificial Sequence
  Antisense oligonucleotide
  RNA (1)..(19)
  DNA (20)..(21)
uguuuuugcauugcguuugtc

SEQ ID NO: 21 - DNA, 21 bp, Artificial Sequence
  Antisense oligonucleotide
  RNA (1)..(19)
  DNA (20)..(21)
gcuuacauauauugucuuutt

SEQ ID NO: 22 - DNA, 21 bp, Artificial Sequence
  Antisense oligonucleotide
  RNA (1)..(19)
  DNA (20)..(21)
aaagacaauauauguaagcgt

SEQ ID NO: 23 - DNA, 21 bp, Artificial Sequence
  .
  RNA (1)..(19)
  DNA (20)..(21)
caagcuaucugcgaauauutt

SEQ ID NO: 24 - DNA, 21 bp, Artificial Sequence
  Antisense oligonucleotide
  RNA (1)..(21)
aauauucgcagauagcuuggg

SEQ ID NO: 25 - DNA, 21 bp, Artificial Sequence
  Antisense oligonucleotide
  RNA (1)..(19)
  DNA (20)..(21)
agaucguagccgcaaccaatt

SEQ ID NO: 26 - DNA, 21 bp, Artificial Sequence
  Antisense oligonucleotide
  RNA (1)..(21)
uugguugcggcuacgaucuca

SEQ ID NO: 27 - DNA, 21 bp, Artificial Sequence
  Antisense oligonucleotide
  RNA (1)..(19)
  DNA (20)..(21)
uccugaagguguccaugaatt

SEQ ID NO: 28 - DNA, 21 bp, Artificial Sequence
  Antisense oligonucleotide
  RNA (1)..(19)
  DNA (20)..(21)
uucauggacaccuucaggatg